(19) United States
(12) Patent Application Publication
(10) Pub. No.: US 2006/0161019 A1
(43) Pub. Date: Jul. 20, 2006

(54) Multiple catalyst system and its use in a high hydrocarbon space velocity process for preparing unsaturated aldehydes and acids

(76) Inventors: Michael Stanley DeCourcy, Houston, TX (US); Peter David Klugherz, Huntingdon Valley, PA (US); Charles Michael Lonzetta, Houston, TX (US); Michael Unton, Bear, DE (US)

Correspondence Address: ROHM AND HAAS COMPANY;PATENT DEPARTMENT, 100 INDEPENDENCE MALL WEST, PHILADELPHIA, PA, 19106-2399, US

(21) Appl. No.: 11/036,007

(22) Filed: Jan. 14, 2005

(51) Int. Cl. C07C 51/16

(52) U.S. Cl. 562/546; 562/547

(57) ABSTRACT

The present invention relates to a multiple catalyst system for preparing unsaturated aldehydes and acids from reactive hydrocarbons at high reactive hydrocarbon space velocity conditions. The present invention also relates to a process for preparing unsaturated aldehydes and acids from reactive hydrocarbons using the multiple catalyst system at high reactive hydrocarbon space velocity conditions. In one embodiment, the multiple catalyst system is utilized in a vapor phase catalytic oxidation reaction process which produces acrolein and acrylic acid from propylene at high reactive hydrocarbon space velocity conditions.

### FIELD OF THE INVENTION

The present invention relates to an improved multiple catalyst system for producing unsaturated aldehydes and acids, including (meth)acrolein and/or (meth)acrylic acid, from reactive hydrocarbons at high reactive hydrocarbon space velocity conditions. The present invention also relates to a process for producing unsaturated aldehydes and acids, including (meth)acrolein and/or (meth)acrylic acid, from reactive hydrocarbons utilizing the improved multiple catalyst system and having a high reactive hydrocarbon space velocity.

### BACKGROUND OF THE INVENTION

Unsaturated aldehydes and carboxylic acids are important commercial chemicals. Of particular importance are (meth)acrylic acid and (meth)acrolein. The highly reactive double bond and acid/aldehyde functionality of (meth)acrylic acid and (meth)acrolein make them suitable reactants with other monomers to produce commercially important polymers. Unsaturated acids/aldehydes are useful reactants in esterification producing commercially important (meth)acrylate esters.

Although the invention is generally described herein with regard to the preparation of acrolein and/or acrylic acid, it is applicable to the preparation of other unsaturated aldehydes and acids, including (meth)acrolein and (meth)acrylic acid.

The preparation of acrylic acid from propylene generally proceeds in a two-step vapor phase catalytic oxidation reaction. In the first step (“step 1”, “stage 1”, or “first stage”), propylene is oxidized using a catalyst in the presence of oxygen and, optionally, other gases, including inert gases and water vapor, to produce acrolein according to equation (I):
C₃H₆+O₂ [symbol] C₂H₃CHO+H₂O+heat (I).
The acrolein is then oxidized, in a second step (“step 2”, “stage 2”, or “second stage”), using a second catalyst in the presence of oxygen, inert gases and water vapor to form acrylic acid according to equation (II):
C₂H₃CHO+½O [symbol] C₂H₃COOH+heat (II).

The aforesaid two-step vapor phase catalytic oxidation of propylene to acrylic acid is generally performed using either tandem reactors wherein a separate reactor is utilized for each step (or stage) or by utilizing one reactor to perform both steps (stages). The preparation of (meth)acrolein and (meth)acrylic acid from isobutylene proceeds in a similar manner.

Several problems are attendant with increasing the capacities of known (meth)acrolein and (meth)acrylic acid manufacturing processes. For example, in the preparation of acrolein and/or acrylic acid, when propylene in the reactant composition is fed at high propylene concentrations or capacities, the two-step exothermic oxidation of propylene to acrylic acid can proceed too quickly, become difficult to control, or can become a runaway reaction. Catastrophic failures could occur. Increased heat production from increased reaction rates can also result in hot spot formation in the reactor system. Hot spots are maximums through which the reaction temperature of a particular reaction passes as the reactants flow through a reactor tube. Hot spots can result in shortened catalyst service life and reduced selectivity.

It is a constant goal of manufacturers to maximize productivity from manufacturing processes and to operate reaction processes under high load conditions and at high reactive hydrocarbon space velocity.

### BRIEF SUMMARY OF THE INVENTION

The multiple catalyst system of the present invention achieves the goal of catalyzing reactions of reactive hydrocarbons under high load conditions, increased reactant concentrations and increased space velocity. Processes employing the multiple catalyst system of the present invention, under high load conditions, are controllable and the multiple catalyst system may be regenerated. In one embodiment, this invention is utilized to manufacture (meth)acrolein and/or (meth)acrylic acid.

In one embodiment of the present invention, the multiple catalyst system, comprises at least one first catalyst and at least one second catalyst which are capable of catalyzing the oxidation of a reactive hydrocarbon to its corresponding unsaturated carboxylic acid, said at least one first catalyst being capable of being regenerated by exposure to an oxygen containing gas and being capable of catalyzing the oxidation of the reactive hydrocarbon to at least a second reactive hydrocarbon and having a composition expressed by the general formula:
MoₐBi_{b}Fe_{c}A_{d}EₑOₓ,

wherein O is oxygen; A is at least one element selected from among Ni and Co; E is at least an element selected among alkali metal elements or alkaline earth metal elements, Tl, P, Te, Sb, Sn, Ce, Pb, Nb, Mn, As, Zn, Si, B, Al, Ti, Zn and W; and wherein a, b, c, d, e and x are the relative atomic ratios of the respective elements Mo, Bi, Fe, A, E and O, where a is 12, b is 0.1-10, c is 0.1-20, d is 1-20, e is 0-30, and x is a positive numerical value determined by the oxidation state of the other elements; and

said at least one second catalyst being capable of maintaining its activity levels upon exposure to an oxygen-containing gas and being capable of catalyzing the oxidation of the second reactive hydrocarbon to the corresponding unsaturated carboxylic acid and having a composition expressed by the general formula:
MoₐV_{b}Cu_{c}(W)_{d}(Sb)ₑ(A)_{f}(G)_{g}(Y)ₕOₓ;

wherein A is at least an element selected from among alkali metal elements, and thallium; G is at least one element selected from among alkaline earth metals and zinc; Y is at least one element selected among Nb, Mn, Fe, Co, Ge, Sn, As, Ce, Ti, and Sm; O is oxygen; and wherein a, b, c, d, e, f, g, h, and x are the relative atomic ratios of the respective elements Mo, V, Cu, W, Sb, A, G, Y and O, where a is 12, b is 0.5-12, c is less than or equal to 6, d is 0.2-10, e is positive and less than or equal to 10; f is 0-0.5; g is 0-1; h is positive and less than 6; and x is a positive numerical value determined by the oxidation state of the other elements.

In another embodiment of the present invention, a process for producing an unsaturated carboxylic acid, comprises the steps of providing the aforesaid multiple catalyst system; exposing a reactant feed stream comprising the reactive hydrocarbon to said multiple catalyst system, in the presence of oxygen; catalyzing the oxidation of the reactive hydrocarbon, in the presence of oxygen, to produce a second reactive hydrocarbon using said at least one first catalyst; and catalyzing the oxidation of the second reactive hydrocarbon, in the presence of oxygen, to produce the corresponding unsaturated carboxylic acid using said at least one second catalyst.

In yet another embodiment of the present invention, a method of regenerating a catalyst system, comprises the steps of providing the aforesaid multiple catalyst system disposed in a reactor apparatus employed in the oxidation of a reactive hydrocarbon to its corresponding unsaturated carboxylic acid; oxidizing the reactive hydrocarbon by exposing the reactive hydrocarbon to the multiple catalyst system in the presence of oxygen until the yield of unsaturated carboxylic acid decreases by more than from 0.5% to 10%; discontinuing the oxidation of the reactive hydrocarbon and the oxidation of the second reactive hydrocarbon; and exposing said at least one first catalyst to an oxygen containing gas at a temperature ranging from at least 105° C. to less than or equal to 415° C.

Another embodiment of the present invention includes a method of catalytic vapor phase oxidation, comprising:

providing an oxidation reactor comprising a plurality of tubes disposed in a reactor shell and having at least a first heat transfer zone and a second heat transfer zone, said at least one of said tubes passing through at least said first heat transfer zone and said second heat transfer zone, said at least one of said tubes containing a multiple catalyst system comprising at least a first catalyst and a sequentially disposed second catalyst, said multiple catalyst system being capable of catalyzing the oxidation of a reactive hydrocarbon to its corresponding unsaturated carboxylic acid,
said first catalyst being capable of being regenerated by exposure to an oxygen containing gas and being capable of oxidizing the reactive hydrocarbon to at least a second reactive hydrocarbon and having a composition expressed by the general formula:
   MoₐBi_{b}Fe_{c}A_{d}EₑOₓ,

wherein O is oxygen;

A is at least one element selected from among Ni and Co; E is at least an element selected among alkali metal elements or alkaline earth metal elements, Tl, P, Te, Sb, Sn, Ce, Pb, Nb, Mn, As, Zn, Si, B, Al, Ti, Zn and W; and
wherein a, b, c, d, e and x are the relative atomic ratios of the respective elements Mo, Bi, Fe, A, E and O, where a is 12, b is 0.1-10, c is 0.1-20, d is 1-20, e is 0-30, and x is a positive numerical value determined by the oxidation state of the other elements; and
said second catalyst being capable of maintaining its activity levels upon exposure to an oxygen-containing gas and being capable of oxidizing the second reactive hydrocarbon to the corresponding unsaturated carboxylic acid and having a composition expressed by the general formula:
   MoₐV_{b}Cu_{c}(W)_{d}(Sb)ₑ(A)_{f}(G)_{g}(Y)ₕOₓ;

wherein A is at least an element selected from among alkali metal elements, and thallium;

G is at least one element selected from among alkaline earth metals and zinc;

Y is at least one element selected among Nb, Mn, Fe, Co, Ge, Sn, As, Ce, Ti, and Sm;

O is oxygen; and
wherein a, b, c, d, e, f, g, h, and x are the relative atomic ratios of the respective elements Mo, V, Cu, W, Sb, A, G, Y and O, where a is 12, b is 0.5-12, c is less than or equal to 6, d is 0.2-10, e is positive and less than or equal to 10; f is 0-0.5; g is 0-1; h is positive and less than 6; and x is a positive numerical value determined by the oxidation state of the other elements;

feeding the reactive hydrocarbon and oxygen to said at least one of said plurality of tubes containing the multiple catalyst system at a reactive hydrocarbon space velocity of from 135 hr⁻¹ to 300 hr⁻¹; and

maintaining the temperature of said first heat transfer zone and said second heat transfer zone at a difference of 5° C. or less.

In still another embodiment of the present invention, a catalytic vapor phase oxidation process, comprises:

providing an oxidation reactor comprising a plurality of tubes disposed in a reactor shell and at least first and second heat transfer zones through each of which a heat transfer medium passes; each of said tubes containing a multiple catalyst system comprising at least a first catalyst and a sequentially disposed second catalyst,
said first catalyst being capable of being regenerated by exposure to an oxygen containing gas and having a composition expressed by the general formula:
   MoₐBi_{b}Fe_{c}A_{d}EₑOₓ,

wherein O is oxygen;

A is at least one element selected from among Ni and Co; E is at least an element selected among alkali metal elements or alkaline earth metal elements, Tl, P, Te, Sb, Sn, Ce, Pb, Nb, Mn, As, Zn, Si, B, Al, Ti, Zn and W; and
wherein a, b, c, d, e and x are the relative atomic ratios of the respective elements Mo, Bi, Fe, A, E and O, where a is 12, b is 0.1-10, c is 0.1-20, d is 1-20, e is 0-30, and x is a positive numerical value determined by the oxidation state of the other elements; and
said second catalyst being capable of maintaining its activity levels upon exposure to an oxygen-containing gas and having a composition expressed by the general formula:
   MOₐV_{b}Cu_{c}(W)_{d}(Sb)ₑ(A)_{f}(G)_{g}(Y)ₕOₓ;

wherein A is at least an element selected from among alkali metal elements, and thallium;

G is at least one element selected from among alkaline earth metals and zinc;

Y is at least one element selected among Nb, Mn, Fe, Co, Ge, Sn, As, Ce, Ti, and Sm;

O is oxygen; and
wherein a, b, c, d, e, f, g, h, and x are the relative atomic ratios of the respective elements Mo, V, Cu, W, Sb, A, G, Y and O, where a is 12, b is 0.5-12, c is less than or equal to 6, d is 0.2-10, e is positive and less than or equal to 10; f is 0-0.5; g is 0-1; h is positive and less than 6; and x is a positive numerical value determined by the oxidation state of the other elements;
said multiple catalyst system being capable of effecting the oxidation of a reactive hydrocarbon to a product gas comprising (meth)acrylic acid, said first catalyst being capable of effecting the oxidation of a reactive hydrocarbon to (meth)acrolein and being substantially located in that portion of each tube in contact with the first heat transfer zone, said second catalyst being capable of effecting the oxidation of (meth)acrolein to (meth)acrylic acid and being substantially located in that portion of each tube in contact with the second heat transfer zone; said first and second catalysts being packed within said tubes in such a manner so as to provide a peak-to-salt temperature sensitivity of not more than 9° C.; and
feeding a reactant composition comprising
   (i) at least one reactive hydrocarbon, and
   (ii) oxygen
      into said oxidation reactor at a space velocity of from 135 hr⁻¹ to 300 hr⁻¹.

Another embodiment of the present invention relates to a catalytic vapor phase oxidation process, comprising:
providing a first oxidation reactor comprising a plurality of tubes disposed in a reactor shell having an interior, the interior of the reactor shell being divided into at least a first heat transfer zone through each of which a heat transfer medium passes; each of said tubes containing at least one first catalyst, said at least one first catalyst being packed in a manner so as to provide a peak-to-salt temperature sensitivity of not more than 9° C., said at least one first catalyst being capable of being regenerated by exposure to an oxygen containing gas and having a composition expressed by the general formula:
   MoₐBi_{b}Fe_{c}A_{d}EₑOₓ,

wherein O is oxygen;

A is at least one element selected from among Ni and Co; E is at least an element selected among alkali metal elements or alkaline earth metal elements, Ti, P, Te, Sb, Sn, Ce, Pb, Nb, Mn, As, Zn, Si, B, Al, Ti, Zn and W; and
wherein a, b, c, d, e and x are the relative atomic ratios of the respective elements Mo, Bi, Fe, A, E and O, where a is 12, b is 0.1-10, c is 0.1-20, d is 1-20, e is 0-30, and x is a positive numerical value determined by the oxidation state of the other elements; and

feeding said first reactant composition comprising
(i) at least one first reactive hydrocarbon, and
(ii) oxygen
   into said first oxidation reactor, at a first reactive hydrocarbon space velocity of from 135 hr⁻¹ to 300 hr⁻¹, to contact said first reactant composition with at least one first catalyst to form a first product gas comprising at least one second reactive hydrocarbon and oxygen;

wherein, when each said tubes of said first oxidation reactor comprises a plurality of sequentially disposed reaction zones, each reaction zone after the first reaction zone of each of said tubes has a temperature that is less than 5° C. greater than its immediately preceding reaction zone;

providing a second oxidation reactor comprising a plurality of tubes disposed in a reactor shell, the inside of the reactor shell being divided into at least a first heat transfer zone through each of which a heat transfer medium passes; each of said tubes containing at least one second oxidation catalyst, said at least one second oxidation catalyst being capable of maintaining its activity levels upon exposure to an oxygen-containing gas and being capable of effecting the oxidation of said second reactive hydrocarbon and oxygen to a final product gas comprising (meth)acrylic acid and having a composition expressed by the general formula:
MoₐV_{b}Cu_{c}(W)_{d}(Sb)ₑ(A)_{f}(G)_{g}(Y)ₕOₓ;

wherein A is at least an element selected from among alkali metal elements, and thallium;

G is at least one element selected from among alkaline earth metals and zinc;

Y is at least one element selected among Nb, Mn, Fe, Co, Ge, Sn, As, Ce, Ti, and Sm;

O is oxygen; and

wherein a, b, c, d, e, f, g, h, and x are the relative atomic ratios of the respective elements Mo, V, Cu, W, Sb, A, G, Y and O, where a is 12, b is 0.5-12, c is less than or equal to 6, d is 0.2-10, e is positive and less than or equal to 10; f is 0-0.5; g is 0-1; h is positive and less than 6; and x is a positive numerical value determined by the oxidation state of the other elements, said tubes containing at least one second oxidation catalyst being packed with said at least one second catalyst in such a manner so as to provide a peak-to-salt temperature sensitivity of not more than 9° C.;

feeding said first product gas comprising
(i) at least one second reactive hydrocarbon, and
(ii) oxygen
   into said second oxidation reactor, at a second reactive hydrocarbon space velocity of from 135 hr⁻¹ to 300 hr⁻¹; to contact said first product gas with said at least one second oxidation catalyst to form a final product gas comprising (meth)acrylic acid;

wherein, when each said tube of said second oxidation reactor comprises a plurality of sequentially disposed reaction zones, each reaction zone after the first reaction zone of each of said tubes has a temperature that is less than 5° C. greater than its immediately preceding reaction zone.

### BRIEF DESCRIPTION OF THE DRAWINGS

A more complete understanding of the present invention will be gained from the embodiments discussed hereinafter and with reference to the accompanying drawings, in which like reference numbers indicate like features, and wherein:

FIG. 1 is a single reactor shell reactor suitable for use in connection with the present invention;

FIG. 2 is a simplified schematic representation of a reactor tube having reaction zones A and B;

FIG. 3 is a simplified schematic representation of a reactor tube having reaction zones A, A′, B and B′;

FIG. 4 is a simplified schematic representation of a reactor tube having reaction zones A, A′, C, B, B′;

FIG. 5 is a simplified schematic representation of a reactor tube having reaction zones A, A′, C, B, B′ and illustrating a tube sheet;

FIG. 6 is a simplified schematic representation of a reactor tube having reaction zones A, A′, A″, B, B′ and B″;

FIG. 7 is a simplified schematic representation of one embodiment of a process in accordance with the present invention which employs an single reactor shell (“SRS”) reactor; and

FIG. 8 is a simplified schematic representation of another embodiment of a process in accordance with the present invention which employs a tandem reaction system.

### DETAILED DESCRIPTION OF THE INVENTION

The definitions set forth below apply to the terminology used hereinafter, unless otherwise indicated.

The term “catalyst” means a substance or composition whose presence facilitates one or more chemical reactions without being directly involved in the chemical reactions and is intended to include, without limitation, pure, diluted, blended and mixed catalysts, as well as catalysts provided on support material, or by coating, impregnating and the like. The physical shape of the catalyst is not limited and, therefore, includes, without limitation, pellets, spheres, columns, rings, tablets, etc. The catalysts and their final shapes may be prepared by any suitable method, including, but not limited to, extrusion, coating, vapor deposition, grinding, extraction, etc.

The term “100 percent catalyst” (“100% catalyst”) refers not only to pure catalyst material, but to 100% of a catalyst material as purchased (which includes catalyst, or catalyst on a support material and/or catalyst with impurities). That is 100% catalyst refers to 100% of the catalyst composition, or blend, as purchased, whether it be as neat chemical or with a support material or a diluent.

The term “reactive hydrocarbon” refers to hydrocarbon compositions which react with one another or other reactant materials, such as oxygen, water, etc., and encompasses pure and substituted hydrocarbons, as well as unsaturated hydrocarbons and hydrocarbon derivatives. Examples of reactive hydrocarbons suitable for use with the present invention include, without limitation, propylene, isobutylene and acrolein.

“Standard conditions”, as used herein, which are also referred to as standard temperature and pressure (“STP”), are 32° F. (0° C.) and 14.696 psia (1 atm), with standard volume being 359.0 ft³/lbmole (22.37 m³/kgmole).

The term “oxygen containing gas”, as used herein, means any gas comprising from 0.01% up to 100% oxygen, including for example, air, oxygen-enriched air, pure oxygen, and mixtures of pure oxygen and at least one inert gas, or mixtures thereof. Although the oxygen containing gas may be pure oxygen gas, it is usually more economical to use an oxygen containing gas, such as air, since purity is not particularly required.

It is noted that the combined total volume of all the tubes within a reactor is the sum total of the interior volume of all reactor tubes of the reactor when the tubes are in an empty (unpacked) state.

“Space velocity”, as used herein, means the volume of feed (at standard conditions) per unit time per unit volume of reactor and is a measure of the relationship between the feed rate and the reactor volume in a flow process. Space velocity values provided herein are calculated on three separate bases. Calculations employing each of the different basis result in separately categorized space velocity values. More particularly, three types of space velocity values are provided herein, as follows: total space velocity (“TSV”), reactive hydrocarbon space velocity (“RHSV”), and propylene space velocity (“C3-SV”). Unless they are specified as TSV, RHSV or C3-SV types of space velocity, the space velocity values discussed hereinafter should be understood to be an RHSV value of a gaseous stream.

TSV is determined based upon the total gas feed to a reactor. RHSV is determined based upon all reactive hydrocarbons present in a feed stream to a reactor. C3-SV is determined based upon the propylene fed to a reactor. Typically, C3-SV values are utilized in conjunction with embodiments relating to the production of acrylic acid from propylene. C3-SV is a subset of RHSV calling attention to the propylene concentration of a feed stream.

The following formulaic definitions for TSV, RHSV and C3-SV, as these terms are used hereinafter, are provided for clarity.

$$TSV = \frac{\begin{matrix}( the combined volumetric flow rate of all \\ gases present in a reactor feed at STP )\end{matrix}}{\begin{matrix}( the combined total volume of \\ all of the tubes within a reactor )\end{matrix}}$$

$$RHSV = \frac{\begin{matrix}( the combined volumetric flow rate of all reactive \\ hydrocarbons present in a reactor feed at STP )\end{matrix}}{\begin{matrix}( the combined total volume of \\ all of the tubes within a reactor )\end{matrix}}$$

$$C 3 \text{-} SV = \frac{\begin{matrix}( the volumetric flow rate of propylene \\ present in a reactor feed at STP )\end{matrix}}{\begin{matrix}( the combined total volume of \\ all of the tubes within a reactor )\end{matrix}}$$

RHSV is a measure of the volume of reactive hydrocarbon which encounters a particular volume of catalyst per unit time. When the concentration of the reactive hydrocarbon, or the TSV, is changed, the RHSV and the C3-SV, will change. When the reactive hydrocarbon concentration or the TSV is raised, the RHSV will also increase thereby resulting in higher load conditions on the catalyst.

It is noted that for embodiments wherein substantially the only the reactive hydrocarbon present in the feed is propylene, the C3-SV will be equal to the RHSV.

The term “residence time” as used herein=1/SV.

Unless otherwise stated, references to percentages (i.e., %) are by volume percent (“vol %”) and all temperatures are in degrees centigrade (° C.).

The percent acrolein selectivity (“acrolein selectivity”) is defined as:

$$acrolein selectivity = \frac{\begin{matrix}Number of moles \\ of acrolein produced\end{matrix}}{\begin{matrix}Number of moles \\ propylene converted\end{matrix}} \times 100.$$

The percent yield of acrolein (“acrolein yield”) is defined as:

$$\% yield of acrolein = \frac{\begin{matrix}Number of moles \\ acrolein produced\end{matrix}}{\begin{matrix}Number of moles \\ propylene employed\end{matrix}} \times 100 ,$$

The percent conversion of propylene (“propylene conversion” or simply “conversion”) is defined as:

$$\% conversion of propylene = \frac{\begin{matrix}Number moles \\ propylene converted\end{matrix}}{\begin{matrix}Number of moles \\ propylene employed\end{matrix}} \times 100 ,$$

The percent selectivity of acrylic acid (“AA selectivity”) is defined as:

$$\% selectivity of acrylic acid = \frac{\begin{matrix}Number of moles of \\ acrylic acid produced\end{matrix}}{\begin{matrix}Number of moles \\ propylene converted\end{matrix}} \times 100 ,$$

The percent yield of acrylic acid (“AA yield”) is defined as:

$$\% yield of acrylic acid = \frac{\begin{matrix}Number of moles \\ acrylic acid produced\end{matrix}}{\begin{matrix}Number of moles \\ propylene employed\end{matrix}} \times 100 ,$$

Endpoints of ranges are considered to be definite and are recognized to incorporate within their tolerance other values within the knowledge of persons of ordinary skill in the art, including, but not limited to, those which are insignificantly different from the respective endpoint as related to this invention (in other words, endpoints are to be construed to incorporate values “about” or “close” or “near” to each respective endpoint). The range and ratio limits, recited herein, are combinable. For example, if ranges of 1-20 and 5-15 are recited for a particular parameter, it is understood that ranges of 1-5,1-15, 5-20, or 15-20 are also contemplated and encompassed thereby.

Part per million (ppm) values are based upon weight.

Pressure values are absolute, unless otherwise stated.

The terms “water vapor” and “steam” are synonymous.

An “inert” material is a material which does not participate in, is unaffected by, and/or is inactive in the particular reaction system being discussed. For example, propane (C₃H₈) and nitrogen are considered to be inert in reaction systems that produce unsaturated aldehydes and acids, such as (meth)acrolein and/or (meth)acrylic acid, from propylene by the two-stage vapor phase catalytic oxidation process described hereinabove and further discussed hereinafter.

The term “major amount” means greater than 50 wt % of a total composition. The term “minor amount” means less than 50 wt % of a total composition.

The term “(meth)acrylic acid” encompasses both acrylic acid and (meth)acrylic acid. The term “acrylic acid” encompasses “(meth)acrylic acid” and related/like compounds. The term “(meth)acrylonitrile” encompasses acrylonitrile and methacrylonitrile and the reverse is also true. The term “(methyl)styrene” encompasses both styrene and methylstyrene and the reverse is also true.

A “packing schedule” is the detailed description of the number and length of zones of catalyst and zones of inert material (e.g., interstage, or preheat zone), as well as the relative amount of catalyst in each zone (e.g., as a percentage vs. diluent in the zone's specific mixture) of the reaction system. It is noted that the packing schedule determines, among other things, the number of reaction zones in a particular reactor, as well as the relative activities of each such reaction zone.

The “first stage reaction zone” (“first stage”) is the region within a reactor where the first oxidation step of a two-step vapor phase catalytic oxidation reaction occurs. For example, in the two-step vapor phase catalytic oxidation of propylene to acrylic acid, the oxidation of propylene to acrolein typically occurs primarily in the first stage reaction zone.

The “second stage reaction zone” (“second stage”) is the region within a reactor where the second oxidation step of a two-step vapor phase catalytic oxidation reaction occurs. For example, in the two-step vapor phase catalytic oxidation of propylene to acrylic acid, the oxidation of acrolein to acrylic acid typically occurs primarily in the second stage reaction zone.

A “single reactor shell” reactor (“SRS” reactor), is a reactor in which a single reactor vessel contains a first stage reaction zone and a second stage reaction zone.

A “tandem reaction” system is a reaction system employing more than one reactor vessel. In one embodiment, such a reaction system may comprise two vessels in series, one having the first stage reaction zone and the other having a second stage reaction zone. In other embodiments, each reactor of a tandem reactor system may have more than one reaction zone, and may also include zones where no catalyst is present. In a particular embodiment, a tandem reactor system used to conduct two-step vapor phase catalytic oxidation has a first reaction vessel including a first stage reaction zone and a second reactor vessel including a second stage reaction zone in series with the first reactor vessel.

A “staged air reactor” system, as used herein, means a tandem reactor system with additional air, or other oxygen-containing gas, being introduced to one or more stages, vessels, or reaction zones downstream (after) the first stage, vessel or reaction zone.

The term “multiple catalyst system”, as used herein, means a catalyst system comprising at least two catalysts which are different from one another and are used in the same reaction system. The at least two catalysts are different from one another in at least one of the following ways: molecular composition, or performance characteristics (such as conversion, yield or selectivity). For example, as discussed herein, one embodiment of a multiple catalyst system that is suitable for performing a two-step vapor phase catalytic oxidation reaction would include at least a first catalyst capable of oxidizing a reactive hydrocarbon to a second reactive hydrocarbon, and at least a second catalyst capable of oxidizing the second reactive hydrocarbon to the corresponding unsaturated aldehyde or acid.

The term “catalyst service life” (or simply “service life”), as used herein, refers to the length of time that a given catalyst may be economically used in a process before it requires replacement. This evaluation is based on various factors, including, but not limited to, the cost of catalyst, the minimum desired product yield, product demand, as well as other market factors familiar to persons of ordinary skill in the art.

The term “time on stream” (“TOS”) means the number of operating-service hours that a catalyst has experienced. The TOS of a catalyst is a measure of the total cumulative hours that the catalyst has spent in operational service, including start-up, normal operations, shut-down, purging, regeneration, and may also include periods where the catalyst is not actually catalyzing a reaction.

“Propylene” as used herein includes pure propylene as well as propylene feed stocks. Propylene feed suitable for use in connection with the present invention may be, without limitation, from any source and of any grade suitable for use in a vapor phase oxidation reaction producing acrolein and/or acrylic acid. For example, suitable grades of propylene feed include, but are not limited to, polymer grade (generally greater than or equal to 99% propylene), chemical grade (generally greater than or equal to 94% propylene), and refinery grade (generally greater than or equal to 60% propylene). In a typical embodiment of the invention, the actual propylene content of the propylene feed is greater than or equal to 94% propylene. Chemical grade or refinery grade propylene feed includes combustible materials, such as propane, which are present as impurities. Generally, chemical grade propylene contains up to 6 percent combustible impurities and refinery grade propylene contains up to 40 percent combustible impurities.

Atoms of elements herein are referred to by atomic number, atom name, IUPAC symbol, periodic table of the elements group identification and/or symbol, common group name, group number, group roman numeral symbol, common name and any equivalent or synonymous representations, known to one having ordinary skill in the art.

In addition, abbreviations used throughout this application are as follows:

° C.=degrees Centigrade,

mm=millimeters,

% C₃=vol % of propylene,

O₂/C₃=ratio of oxygen to propylene,

% steam=percent by volume of steam or water vapor,

R1-SV=total space velocity into first stage reaction zone (hr⁻¹),

R1 C₃—SV=propylene space velocity into first stage reaction zone (hr⁻¹),

R2-SV=total space velocity into second stage reaction zone (hr⁻¹),

R2 C₃—SV=propylene space velocity into second stage reaction zone (hr⁻¹), and

AA (lb/hr)/tube=pounds of acrylic acid produced per hour per tube.

Various embodiments of the present invention will now be described in detail.

The present invention relates generally to a multiple catalyst system which comprises at least two catalysts which are capable of catalyzing the two-step vapor phase catalytic oxidation of a reactive hydrocarbon to an unsaturated aldehyde or acid. More particularly, the multiple catalyst system comprises at least a first catalyst capable of oxidizing a first reactive hydrocarbon to a second reactive hydrocarbon, and at least a second catalyst capable of oxidizing the second reactive hydrocarbon to the corresponding unsaturated aldehyde or acid. The at least two catalysts are typically different from one another in composition and/or activity (e.g., conversion, yield, selectivity, etc.).

In general, any catalysts that are capable of catalyzing the two-step vapor phase catalytic oxidation of a reactive hydrocarbon to an unsaturated aldehyde or acid are suitable for use in connection with the present invention. For example, one embodiment of the multiple catalyst system of the present invention is capable of catalyzing propylene to acrylic acid. Although the following discussion will describe the multiple catalyst system as applied to the production of acrylic acid from propylene by two-step vapor phase catalytic oxidation, it will be understood, as previously described, that the present invention is not limited to such application and is suitable for use in the production of other unsaturated aldehydes and acids from other types of reactive hydrocarbons.

Catalysts capable of oxidizing propylene to acrolein are hereinafter referred to as “R1 catalysts” (“R1”, “first stage catalysts”, or “first catalysts”) and are suitable for use with the present invention, as will be discussed in further detail hereinafter. In addition, catalysts capable of oxidizing acrolein to acrylic acid are hereinafter referred to as “R2 catalysts” (“R2”, “second stage catalysts”, or “second catalysts”). Either the R1 catalysts, or the R2 catalysts, or both, are typically, but need not be, mixed metal oxide (“MMO”) compositions.

In one embodiment, the multiple catalyst system of the present invention comprises a first R1 catalyst and a second catalyst which is of the R2 type. Other embodiments are possible wherein the multiple catalyst system comprises more than one R1 catalyst and an R2 catalyst, or alternatively, an R1 catalyst and more than one R2 catalyst. Furthermore, the multiple catalyst system of the present invention may comprise more than one R1 catalyst and more than one R2 catalyst. The foregoing variations, and others, will be readily apparent to persons of ordinary skill in the art based upon the following description.

In particular, an R1 catalyst suitable for use in connection with the multiple catalyst system is capable of being regenerated by exposure to an oxygen containing gas and comprises at least one atom of a group VIB element, at least one atom of a group VA element, and at least two group VIII atoms and is capable of catalyzing the production of acrolein. For example, the R1 catalyst may contain more than one atom of the same group VIII element (e.g., group VIII atoms include Fe, Co, Ni, Ru, Rh, Pd, Os, Ir, Pt). In another embodiment, the R1 catalyst comprises two group VIII atoms which are different elements. In a further embodiment, the R1 catalyst comprises at least Fe, Co or Ni. Yet another embodiment of R1 catalyst includes at least two different atoms selected from Fe, Co or Ni and compounds which are mixture thereof (e.g., FeCo, FeNi, CoFe, CoNi).

Commonly, the R1 catalyst comprises at least one atom of a group VIB element (e.g., Cr, Mo, W, or Unh). In another embodiment the R1 catalyst comprises at least Mo.

In one embodiment, the R1 catalyst comprises at least one atom of a group VA element (e.g., N, P, As, Sb or Bi). Another embodiment of R1 catalyst comprises Mo, Bi, Fe and at least one atom of Ni or Co.

An R1 catalyst optionally includes at least one atom from any of the alkali metal elements of group IA (e.g., H, Li, Na, K, Rb, Cs, or Fr). An R1 catalyst also optionally comprises one or more atoms of elements from groups including: IIIA (e.g., B, Al, Ga, In or Tl) with one embodiment having at least one of Al or TI; IIIB including the elements of the lanthanide series and the actinide series (e.g., Sc, Y, La, Ac, Ce, Pr, Nd, Pm, Sm, Eu, Gd, Th, Dy, Ho, Er, Tm, Yb, Lu, Th, Pa, U, Np, Pu, Am, Cm, Bk, Cf, Es, Fm, Md, No, or Lr) with one embodiment having Ce; VIIB (e.g., Mn, Tc, Re, or Uns); VIA (e.g., O, S, Se, Te, or Po) with one embodiment having Te; IVA (e.g., C, Si, Ge, Sn, or Pb) with one embodiment having at least one of Mn or Pb; VB (e.g., V, Nb, Ta, or Unp) with one embodiment having Nb; IVB (e.g., Ti, Zr, Hf, or Unq) with one embodiment having Ti; IIIB (e.g., Zn, Cd, or Hg) with one embodiment having Zn.

In a typical embodiment, an R1 catalyst is capable of being regenerated by exposure to an oxygen containing gas and its composition is expressed by the general formula:
MOₐBi_{b}Fe_{c}A_{d}EₑOₓ,

wherein O is oxygen;

A is at least one element selected from among Ni and Co; E is at least an element selected among alkali metal elements or alkaline earth metal elements, Tl, P, Te, Sb, Sn, Ce, Pb, Nb, Mn, As, Zn, Si, B, Al, Ti, Zn and W; and

wherein a, b, c, d, e and x are the relative atomic ratios of the respective elements Mo, Bi, Fe, A, E and O, where a is 12, b is 0.1-10, c is 0.1-20, d is 1-20, e is 0-30, and x is a positive numerical value determined by the oxidation state of the other elements.

One embodiment of R1 catalyst comprises 50-75 mole % MoO₃, 5-20 mole % Bi₂O₃, 1-10 mole % Fe₂O₃, and 1-10 mole % SiO₂. Another embodiment of an R1 catalyst includes metal oxides from 1-30 mole %. In one embodiment, at least one cobalt oxide is present in a range from about 10 mole % to about 30 mole %. In another embodiment, at least one nickel oxide is present in a range from about 1 mole % to about 10 mole %.

An R2 catalyst is capable of maintaining its activity levels upon exposure to an oxygen-containing gas and comprises at least one atom of a group VIB element (e.g., Cr, Mo, W. or Unh), at least one atom of a group VB element (e.g., V, Nb, Ta, or Unp), at least one atom of a group IB element (e.g., Cu, Ag, or Au), and a group VA element (e.g., N, P, As, Sb or Bi).

An R2 catalyst suitable for use in connection with the multiple catalyst system comprises one or more atoms of elements from groups including: IA (alkali metal element, e.g., H, Li, Na, K, Rb, Cs, or Fr); IIA (alkali earth metal element, e.g., Be, Mg, Ca, Sr, Ba, or Ra); IIIB which also encompasses elements of the lanthanide series and the actinide series (e.g., Sc, Y, La, Ac, Ce, Pr, Nd, Pm, Sm, Eu, Gd, Tb, Dy, Ho, Er, Tm, Yb, Lu, Th, Pa, U, Np, Pu, Am, Cm, Bk, Cf, Es, Fm, Md, No, or Lr); IVB (e.g., Ti, Zr, Hf, or Unq); VB (e.g., V, Nb, Ta, or Unp); VIB (e.g., Mn, Tc, Re, or Uns); VIII (e.g., Fe, Co, Ni, Ru, Rh, Pd, Os, Ir, Pt); IIB (e.g., Zn, Cd, or Hg); IIIA (e.g., B, Al, Ga, In or TI); IVA (e.g., C, Si, Ge, Sn, or Pb); VA (e.g., N, P, As, Sb, or Bi); or VIA (e.g., O, S, Se, Te, or Po).

In one embodiment, an R2 catalyst comprises Mo, V, Cu, W and Sb. Another embodiment of the R2 catalyst comprises antimony present in an amount less than about 5 wt % with a concentration of less than 3% typical and less than 1% in another embodiment.

One embodiment of an R2 catalyst comprises MoO₂ and MoO₃ either individually (i.e., MoO₂ without MoO₃; or MoO₃ without MoO₂) or in combination (i.e., MoO₂ and MoO₃).

In a typical embodiment, an R2 catalyst composition is expressed by the general formula:
MOₐV_{b}Cu_{c}(W)_{d}(Sb)ₑ(A)_{f}(G)_{g}(Y)ₕOₓ;

wherein A is at least an element selected from among alkali metal elements, and thallium;

G is at least one element selected from among alkaline earth metals and zinc;

Y is at least one element selected among Nb, Mn, Fe, Co, Ge, Sn, As, Ce, Ti, and Sm;

O is oxygen; and

wherein a, b, c, d, e, f, g, h, and x are the relative atomic ratios of the respective elements Mo, V, Cu, W, Sb, A, G, Y and O, where a is 12, b is 0.5-12, c is less than or equal to 6, d is 0.2-10, e is positive and less than or equal to 10; f is 0-0.5; g is 0-1; h is positive and less than 6; and x is a positive numerical value determined by the oxidation state of the other elements.

R1 and R2 catalysts optionally comprise a variety of, mechanical and structural types, properties, shapes, compositions, configurations and sizes. R1 and R2 catalysts may be loose, structured, supported, applied as coatings, blended, diluted, doped, activated, inactive, or of any variety or condition which allows an R1 or R2 catalyst to be utilized. R1 and R2 catalysts may be of any variety compatible with the reaction process in which they are employed.

Catalyst size is varied in normal practice in accordance with the reactor design. The diameters of the R1 catalysts and the R2 catalysts generally range from about 2-15 mm. In one embodiment, a reactor having 1 inch internal diameter (ID) reactor tubes utilizes 5 mm diameter catalyst pellets. One embodiment of a tandem reactor design incorporates 1.5 inch ID tubes for catalyst containment. Typically where a reactor incorporates 1.5 inch ID reactor tubes, 7 mm diameter catalyst pellets are utilized. In a typical embodiment, the geometric relationship between reactor tube's internal diameter to R1 or R2 catalyst diameter in one embodiment optionally conforms to the mathematical relationship:
Diameter Of Catalyst Pellet=(0.2) (Internal Diameter of the Reactor Tube).

One embodiment employs catalyst pellets of R1 and/or R2 types which are formed by extrusion of the catalyst material through an “0” shaped opening and cut to form cylindrical objects.

In one embodiment, an R1 catalyst is grayish brown in color and has a hollow cylindrical shape. In another embodiment an R1 catalyst is a cylindrical catalyst pellet of dull, red-brown color with an axial pathway through the cylinder and an appearance similar to fired molding clay.

The specific gravity of an R1 catalyst at 25° C. typically ranges from about 0.70 to about 1.0, as compared to water which has a specific gravity of 1.0 at 25° C.

In one embodiment, an R2 catalyst is a spherical pellet of dull, black color and has the appearance of a being made of, or coated with, graphite. In an embodiment where the R2 catalyst is coated on to a conventional support material, the coating thickness ranges from 1-5 microns, or to greater thicknesses such as 1 mm, 5 mm, 10 mm, or thicker. Any coating thickness they may be employed, typically the coating thickness is less than 10 mm. One embodiment has a coating of 10 microns to 2 mm.

The specific gravity of an R2 catalyst at 25° C. is typically about 1.2, as compared to water which has a specific gravity of 1.0 at 25° C.

One or both of the R1 and R2 catalysts suitable for use with the multiple catalyst system of the present invention may be in pure undiluted form, in 100% catalyst form, or they may be diluted (i.e., blended) with other catalysts, inert substances and/or support materials. Furthermore, any catalyst suitable for the vapor phase catalytic oxidation of propylene to acrolein and acrolein to acrylic acid may be used along with, mixed with, or in combination with R1 or R2 catalysts used in the multiple catalyst system of the present invention.

For example, without limitation, an R1 catalyst may be diluted to 50 wt % to 80 wt % of an R1 catalyst concentration. A 66 wt % to 70 wt % of an R1 catalyst concentration may also be employed. As will be understood by persons of ordinary skill in the art, the mixture of R1 catalyst and diluent material is less active than pure R1 catalyst. Dilution of an R1 or R2 catalyst can be used as a reactor control factor.

An R2 catalyst may be diluted to 60 wt % to 90 wt % of an R2 catalyst concentration. A 70 wt % to 87 wt % of an R2 catalyst concentration may also be employed. As will be understood by persons of ordinary skill in the art, the mixture of R2 catalyst and diluent material is less active than pure R2 catalyst.

The R1 and R2 catalysts of the multiple catalyst system are typically charged, or loaded, into one or more reactors during use. Reactors of the present invention include, but are not limited to SRS, tandem reactors, staged-air tandem batch, semi-batch, continuous stirred tank reactors, flow, packed bed, floating bad, mixed-bed, shell and tube, as well as any equipment capable of supporting a reaction catalyzed by the catalyst system disclosed herein. In one embodiment, for example, a suitable oxidation reactor would be any oxidation reactor suitable for the manufacture of an unsaturated aldehyde or acid.

One embodiment of the present invention comprises a pipe reactor which employs a pipe containing an R1 catalyst and an R2 catalyst, which are separated from one another by non-catalyst materials. In a one embodiment, for example, the R1 catalyst and R2 catalyst may be separated by Raschig rings. In another embodiment, a reactor system is designed to accommodate a two-stage catalyst system where R1 is the first stage catalyst and R2 is the second stage catalyst.

SRS reactors and tandem reaction systems are typically employed in reaction processes which utilize the multiple catalyst system of the present invention.

An SRS reactor provides the ability to operate an oxidation reaction at a variety of different temperatures, or to run more than one oxidation reactions, each at a different temperature, in a single reactor. A single reactor system may be utilized, for instance, when different temperature zones are required or desired in a vapor phase oxidation reaction.

In one embodiment, the oxidation reactor is an SRS reactor which comprises a shell and multiple-tube heat exchange reactor, having a plurality of tubes disposed in a shell through which at least two heat transfer medium circuits pass. Specifically, the interior region of the reactor shell is divided into at least a first heat transfer zone and a second heat transfer zone. The tubes run longitudinally from the top portion to the bottom portion of the reactor through the heat transfer zones and across a perforated tubesheet. It is understood that the reactor optionally contains one or more perforated tubesheets which divide the reactor into multiple (at least two) heat transfer zones.

For example, an SRS system may be utilized in the two-step oxidation of propylene to acrylic acid, wherein propylene is oxidized to acrolein in a first stage at a particular temperature range, and wherein the acrolein in turn passes to the second stage and is oxidized (optionally, at a different temperature range) to acrylic acid. Each of said stages optionally is further divided into two or more sequential reaction zones, each of which may be maintained at a desired temperature. Typically, the temperature of each subsequent reaction zone in a stage is less than 5° C. higher than the temperature of the immediately preceding reaction zone in the stage. In some embodiments, however, the temperature of each subsequent reaction zone in a stage may be greater than 5° C. higher than, or even greater than 10° C. higher than, the temperature of the immediately preceding reaction zone in the stage. Typically, each stage may be divided into two sequential reaction zones.

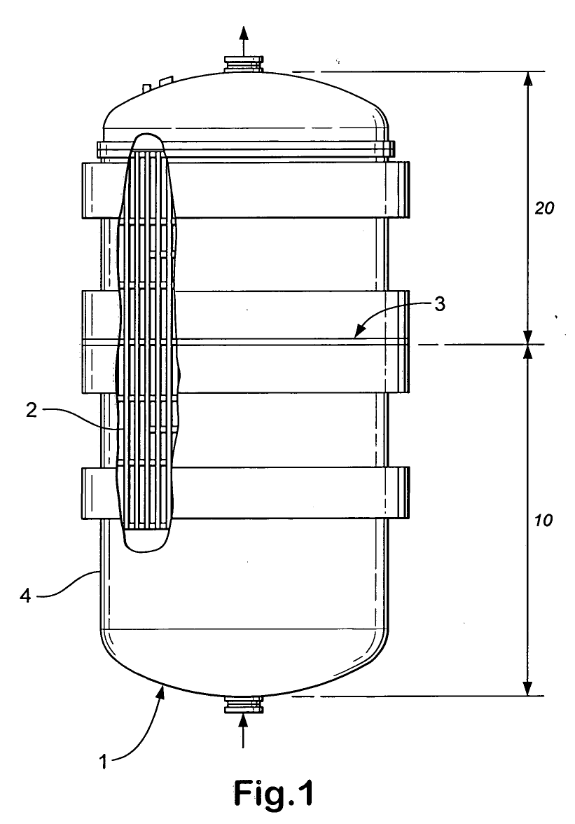

FIG. 1 (FIG. 1) illustrates a single reactor shell (SRS) reactor (1), which is a shell-and-tube type reactor. Like conventional SRS reactors, the SRS reactor (1) is adapted for heat transfer and may comprise from 1 to 100,000, or more, tubes (2). The use of 25,000-50,000 tubes is typical. Typically, an oxidation reactor, such as the SRS reactor (1), will contain greater than 5,000 tubes and greater than 25,000 tubes is common. In one embodiment, the SRS reactor contains greater than 15,000 tubes. Another embodiment might suitably contain greater than 20,000. In an alternative embodiment, the SRS reactor (1) may contain greater than 30,000 tubes. The tubes (2) utilized are those generally known and used in the art for containing catalyst in reactors for operating catalytic reactions. The tubes (2) may be arranged in any suitable arrangement known in the art and may comprise any materials compatible to the particular reaction process to be operated in the SRS reactor (1). A detailed explanation of a typical SRS reactor (1) which would be suitable for use in connection with the multiple catalyst system of the present invention is disclosed in U.S. Pat. No. 6,384,274, which is hereby incorporated herein in its entirety.

As illustrated in FIG. 1, the SRS reactor (1) has one or more tubes (2) disposed in the interior region of the shell (4). The tubes (2) pass through at least one perforated tube sheet (3) which divides the inside of the reactor shell into a first heat transfer zone associated with a first reaction zone (10) and a second heat transfer zone associated with a second reaction zone (20). Alternatively, where the SRS reactor (1) includes more than one perforated tube sheet (not shown), additional heat transfer zones and associated reaction zones would be created within the SRS reactor (1). The tubes (2) typically pass through two or more heat transfer zones and their associated reaction zones within the SRS reactor. The process conditions within any given tube (2) along its length are dependent, at least in part, upon conditions of the particular portion of a heat transfer zone through which it passes.

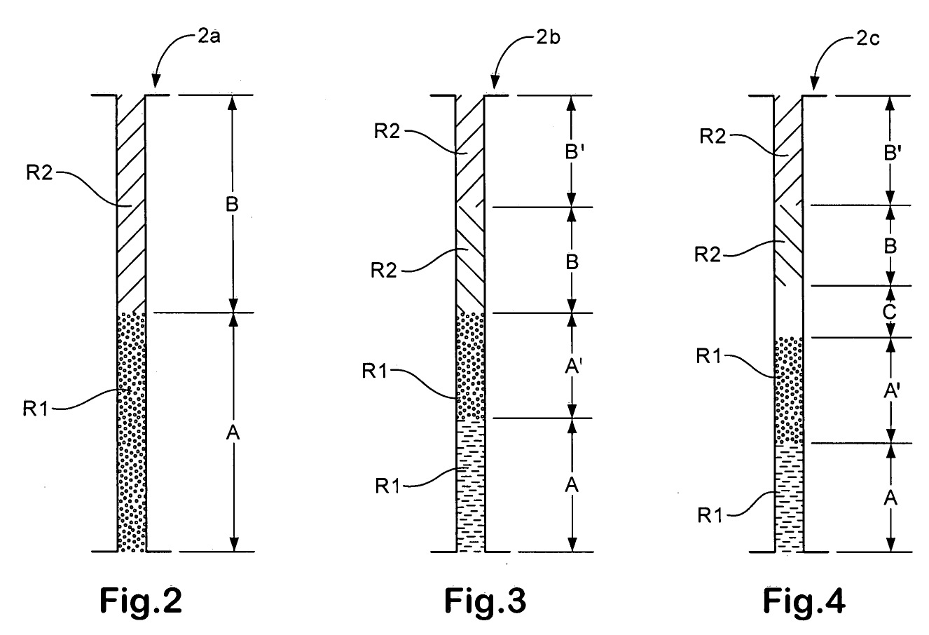

FIG. 2 illustrates an embodiment of a tube (2a) which contains a first reaction zone A and a second reaction zone B. The first reaction zone A has an R1 catalyst disposed therein which is at least capable of catalyzing the oxidation of propylene to acrolein. The second reaction zone B has an R2 catalyst disposed therein which is at least capable of catalyzing the oxidation of acrolein to acrylic acid. More particularly, the R1 catalyst is disposed within at least one of the tubes (2a) passing through the first reaction zone A and the R2 catalyst is disposed within at least one of the tubes (2a) passing through the second reaction zone B. It should be noted that, although the embodiment of FIG. 2 represents the first reaction zone A as being disposed in the tube (2a) ‘beneath’ the second reaction zone B, with process flow proceeding upward from reaction zone A to zone reaction B (illustrating what is known herein as an ‘upflow’ tube configuration), it is within the scope of the present invention to, alternatively, dispose the first reaction zone A ‘above’ the second reaction zone B, with process flow proceeding downward from reaction zone A to reaction zone B (illustrating what is known herein as a ‘downflow’ tube configuration). Furthermore, it is envisioned that in some embodiments of the present invention, it may be advantageous to orient the tube (2a) in a wholly different way—for example, in a horizontal configuration, with process flow proceeding from right-to-left or from left-to-right. The selection of a specific tube orientation may take into account various economic and/or operability considerations, such as, for example, to simplify process piping runs, improve maintenance access to equipment, or to address other equipment-siting issues and is within the ability of one of ordinary skill in the art of chemical process design. It will be apparent to one of ordinary skill in the art that the physical orientation of the tube (2a) does not materially change the operation of the present invention, as long as the first reaction zone A is nonetheless disposed upstream of the second reaction zone B and the two-step oxidation reaction proceeds from reaction zone A to reaction zone B as previously described. FIG. 3 illustrates an embodiment of a tube (2b) having multiple first reaction zones A and A′, each of which contain at least one R1 catalyst disposed therein which is capable of catalyzing the oxidation of propylene to acrolein. Where more than one R1 catalyst is used in each of reaction zones A and A′, the R1 catalysts may optionally have the same or different compositions, or activity characteristics (e.g., selectivity, conversion, or yield). The tube (2b) also has multiple second reaction zones B and B′ each of which contain at least one R2 catalyst disposed therein which is capable of catalyzing the oxidation of acrolein to acrylic acid. Where more than one R2 catalyst is used in each of reaction zones B and B′, the R2 catalysts may optionally have the same or different compositions, or activity characteristics (e.g., selectivity, conversion, or yield). In the embodiment shown in FIG. 3, the multiple first reaction zones A and A′ are upstream of the multiple second reaction zones B, and B′.

In further embodiments, it is possible for each tube (2, 2a, 2b) to have reaction zones in other configurations, such as, without limitation, reaction zones A, A′ and B; or reaction zones A, A′, and B′; or reaction zones A, B, and B′; or reaction zones A′, B, and B′; or other combinations of reaction zones. The reaction zones and catalyst sections having an R1 and/or an R2 catalyst can overlap, alternate, intermingle or be sequential. Further, inert materials may be utilized between different catalyst types, to separate zones, or may be mixed with one or more catalysts. These variations will be readily understood by persons having ordinary skill in the art.

For example, FIG. 4 illustrates an embodiment of a tube (2c) similar to the tube (2b) of FIG. 3 and having an additional zone C (hereinafter referred to as “reaction zone C”, or “quenching zone C”) which may have no catalyst, less catalyst, or a different catalyst composition (for example, 0 wt %-75 wt % catalyst mixed with diluent material) than zones A, A′, B, or B′ and may be disposed in the tube (2c) between two reaction zones of a tube (2c) (for example, without limitation, between reaction zones A and B, or between reaction zones A′ and B, or between reaction zones A and A′, or between reaction zones B and B′). Generally, quenching zone C is less than 10 percent of the total length of the tube and has no catalyst. In one embodiment, quenching zone C is from 100 mm to 1000 mm in length, and in still another embodiment quenching zone C is from 350 mm to 650 mm in length. In addition, although not shown in the accompanying figures, more than one quenching zone C may exist in the tube (2c).

The quenching zone C is typically packed with an inert material (comprising for example, without limitation, alumina, mullite, carborundum, steel (including stainless steel), copper, aluminum and ceramic) of high surface area, and having heat transfer enhancing shape. Typical packing shapes include, e.g., Raschig rings, spheres, cylinders, rings, small pieces, filaments, meshes and ribbons.

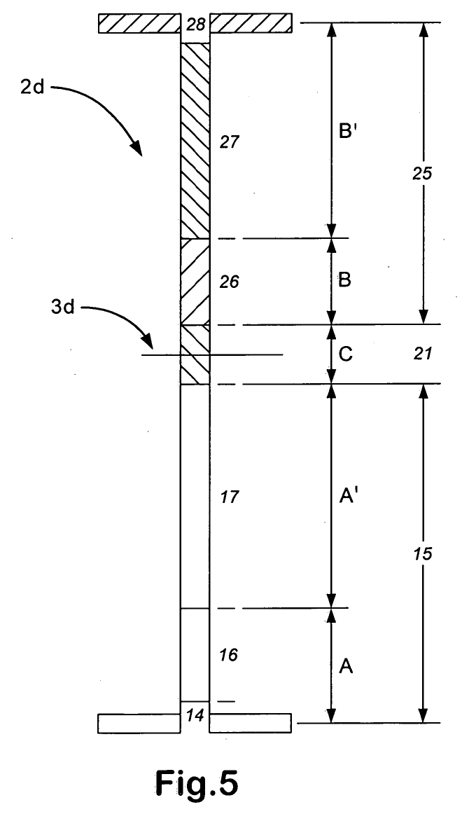

FIG. 5 illustrates an embodiment of a tube (2d) having a first stage (15) including multiple reaction zones A (16), A′ (17), and a second stage (25) including multiple reaction zones, B (26), B′ (27), as well as a quenching zone C (21) which is disposed between reaction zones A′ (17) and B (26). In this embodiment, the tube (2d) passes through the perforated tubesheet (3d) which divides the first stage (15) from the second stage (25) at quenching zone (C) (21). During operation of the oxidation reaction, the reaction gases flow into reaction zone A of the first stage (15), at the bottom of the reactor. The first layer (14) in the bottom of the tube (2d) comprises inert diluent material, which may be, for example, ceramic balls or cylinders. As the reaction gases pass through the first layer (14), preheating occurs and the temperature of the reaction gases is raised to near the temperature of the heat transfer medium before it enters reaction zone A (16). At least one R1 catalyst is disposed within reaction zone A (16). A diluent material, or other catalyst, may be mixed with the at least one R1 catalyst. Suitable diluent materials include, without limitation, one or more of the following: silicon dioxide, silicon carbide, silicon nitride, silicon boride, silicon boronitride, aluminum oxide (alumina), aluminosilicate (mullite), aluminoborosilicate, carborundum, carbon-fiber, refractory fiber, zirconium oxide, yittrium oxide, calcium oxide, magnesium oxide, magnesium oxide-aluminosilicate (cordite), and clay based materials (e.g., Denstone™ line of catalyst supports by Norton Chemical Process Products Corp., of Akron, Ohio).

The reaction gases flow from reaction zone A (16) into the subsequent reaction zone A′ (17), which may have a greater catalytic activity than zone A (16). Reaction zone A′ (17) may be longer than reaction zone A (16). In one embodiment reaction zone A′ (17) is at least 50% longer than reaction zone A (16). In one embodiment, the ratio of the lengths of reaction zones A′/A is in a range from 1.0:1 to 3.0:1, in another embodiment, the ratio of the lengths of reaction zones A′/A ranges from 1.5:1 to 2.5:1. The R1 catalysts disposed in either or both of reaction zones A (16) and A′ (17) may be mixed with one or more diluent materials.

Reaction gases exiting reaction zones A (16) and/or A′ (17) enter the quenching zone C (21). The quenching zone C (21) may contain only catalyst, only inert material, or a mixture of catalyst and inert material. Where the quenching zone C (21) contains catalyst, the catalyst may be at least one R1 catalyst, or at least one R2 catalyst, or any desired mixture of catalysts. Optionally, the quenching zone C (21) may include a baffle and an inert substance which only partially fills quenching zone C (21). Some cooling of the reaction gases occurs in the quenching zone C (21).

Reaction gases exiting the quenching zone C (21) next enter reaction zone B (26) of the second stage (25). The reaction zone B (26) has at least one R2 catalyst disposed therein which catalyzes the oxidation of acrolein to acrylic acid. The R2 catalyst disposed in reaction zone B (26) may be mixed with a diluent or other catalyst, as described hereinabove in connection with the R1 catalyst.

From reaction zone B (26), the reaction gases flow into reaction zone B′ (27), which may have a higher catalytic activity than reaction zone B (26). The reaction zone B′ (27) is optionally longer than reaction zone B (26), for example, at least 50% longer. Typically, the ratio of the lengths of the reaction zones B′/B is in a range of from 1.0:1 to 3.0:1; in some embodiments, the B′/B ratio may be in a range of from 1.5:1 to 2.5:1. The R2 catalyst disposed in reaction zone B′ may be mixed with a diluent material or other catalyst, as described hereinabove in connection with the other reaction zones A, A′ and B.

A mixed product gas, containing acrylic acid and other compounds, flows out of reaction zone B′ (27) and, optionally, through a second layer (28) which may also contain inert diluent material. It should be understood that the inert material in the first and second layers (14), (28) may be the same or different (i.e., composition, shape, diameter) from one another and may also be the same or different from the shape and diameter of the catalysts in adjacent reaction zones A (16), B′ (27), respectively.

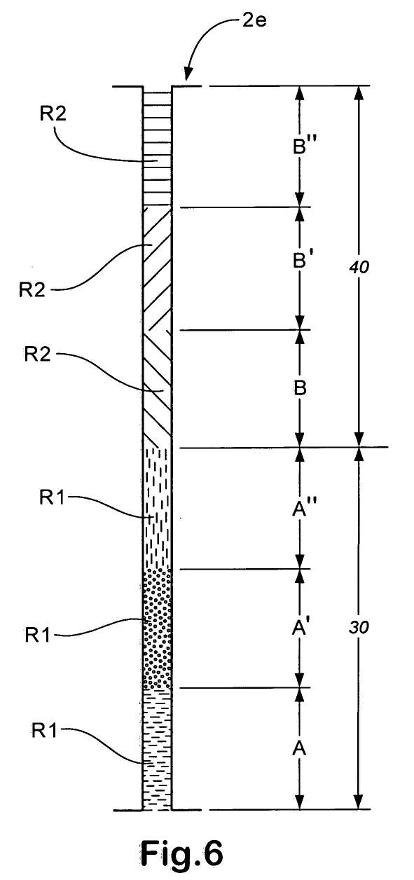

FIG. 6 illustrates another embodiment of a tube (2e) wherein the first reaction zone (30) and the second reaction zone (40) each optionally have an additional reaction zone A″ or B″, respectively. The sequence of the reaction zones is not critical and they may be sequenced in any order or combination desired (e.g., A, A″, A′; B, B″, B′; A′, A, A″, B′, B, B′″ and the like).

In another embodiment, reactions zones may be designed so that each tube (2e) has, for example, reaction zones A, A′ and B; or reaction zones A, A′, and B′; or reaction zones A, B, and B′; or reaction zones A′, B, and B′.

In a further embodiment similar to that shown in FIG. 6, a reaction zone A″ containing 0 wt % to 10 wt % catalyst may be disposed between two other reaction zones A or A′ in each tube (2e). Generally, reaction zone A″ is less than 10% of the total length of the tube (2e). For instance, in one embodiment reaction zone A″ is in a range of from 100 to 1000 mm in length and, in another embodiment, reaction zone A″ is in a range of from 350 to 650 mm in length.

A catalyst charge optionally comprises one or more catalysts (e.g., R1 and/or R2 type catalysts), varying by source, composition, mixture of catalyst types, activity, dilution, preparation methods (calcining, etc), geometry, and/or shape. Generally the first reactor section, or first stage, is the section that contains at least one R1 catalyst and the second reactor section, or second stage, contains at least one R2 catalyst.

The multiple catalyst system of the present invention may comprise non-catalyst materials (e.g., inerts, diluents, packings, internals, and the like). In one alternative embodiment, for example, R1 and R2 catalysts may be disposed in layers such that at least a first layer having an R1 catalyst and at least a second layer having an R2 catalyst with Raschig rings placed between the first and second layers are disposed within one or more of the tubes. The Raschig rings, optionally, are hollow cylinders which can be either 3/16 inch in diameter or ¼ inch diameter and approximately ¼ inch in length. One embodiment utilizes Raschig rings of ¼ inch diameter. The Raschig rings may be pieces of cut stainless steel tubing, ceramic, or other non-reactive material. The Raschig rings may be magnetic.

One embodiment of a two reaction zone SRS reactor configuration utilizes 66 wt %-75 wt % of an R1 catalyst mixed with inert diluent material in a stage one first reaction zone and an undiluted 100 wt % of an R1 catalyst in a stage one second reaction zone. The two zone SRS reactor typically utilizes 70 wt %-75% of an R2 catalyst mixed with inert diluent material in a second stage first reaction zone and an undiluted 100 wt % of an R2 catalyst in a second stage second reaction zone.

In acrylic acid production, the catalyst may be packed into a reactor vessel in any suitable manner. In one embodiment, the tubes of at least one rector vessel include one or more catalysts which individually, or in combination, are capable of catalyzing a reaction producing acrylic acid from acrolein, or of catalyzing reaction producing acrolein from propylene.

One embodiment of a reaction process in accordance with the present invention (not shown) utilizes the multiple catalyst system and an SRS reactor having a tube configuration similar to that shown in FIG. 3 for the production of acrylic acid from propylene. More particularly, the SRS reactor comprises reaction zones A and A′ through which a plurality of tubes pass. The tubes contain at least one R1 catalyst and at least one R2 catalyst, as described hereinabove, which individually or in combination are capable of effecting the oxidation of propylene to acrylic acid. In one embodiment, reaction zones A and A′ optionally have different catalytic activities for converting propylene to acrolein or for converting acrolein to acrylic acid.

There are a number of different methods, which are known in the art, for creating reaction zones (i.e., reaction zones A and A′ or reaction zones B and B′) having different activities. Such methods include, but are not limited to, the following: varying the dilution of the catalysts packed into the reaction zones (i.e., varying the packing schedule, as described above), utilizing catalysts having differing activities, modifying reaction control variables such as temperatures, pressure or flow rates, or any combination of these. More particularly, catalysts having differing activities may be obtained by varying the kind and/or amount of metal constituents or by varying the calcining conditions (i.e., temperature, time, etc.) of the catalysts during their preparation. In addition, catalysts of the same or different composition may also be prepared such that they are comprised of differently sized and/or differently shaped particles or pieces, whereby they will have different occupying volumes. Where catalysts of different activities are used to pack reaction zones, it is often desirable to pack adjacent reaction zones starting with catalyst of lesser activities such that the catalytic activity of the reaction section (i.e., the first stage reaction section or the second stage reaction sections) increases from inlet end toward the outlet. A discussion of various such methods for creating reaction zones having variable activities is provided in U.S. Patent Application Publication No. US 2001/0021789, which is hereby incorporated herein by reference. Thus, catalysts may be selected, produced and packed into the reaction zones in a manner which controls the activities of the reaction zones. The particular manner in which catalysts are selected and packed into reaction zones is sometimes referred to as the “packing schedule” of a particular tube or reaction section and is one method for controlling various process characteristics, including but not limited to, the useful life of the catalyst, the conversions and yields of the process, and the costs of the catalyst.

In an alternative embodiment, each of the tubes contains at least two catalysts at least capable of catalyzing the oxidation of propylene to acrolein and at least capable of catalyzing oxidation of acrolein to acrylic acid. Such catalysts may be packed in the tubes so as to overlap, or be intermingled, or be sequentially disposed within the tubes. In one embodiment, the reactants contact a first catalyst capable of effecting the oxidation of propylene to acrolein and then a second catalyst capable of effecting the oxidation of acrolein to acrylic acid.

In an alternative embodiment, each of the tubes include at least two catalysts at least capable of catalyzing oxidation of propylene to acrolein and at least capable of catalyzing the oxidation of acrolein to acrylic acid. In one embodiment, each of the tubes contain reaction zones A and A′ which respectively contain one or more catalysts at least capable of catalyzing oxidation of propylene to acrolein and reaction zones B and B′ which respectively contain one or more catalysts at least capable of catalyzing the oxidation of acrolein to acrylic acid. In one embodiment, reaction zones A and A′ have different catalytic activities for converting propylene into acrolein and reaction zones B and B′ have different catalytic activities for converting acrolein to acrylic acid. As discussed above, such differing activity may be achieved by catalyst dilution, by using different catalysts having different catalytic activity, or by temperature control.

Reaction zones may include high surface area material with a heat transfer enhancing shape which is inert to and stable in the reaction system. Suitable examples of inert materials include, alumina, alundum, mullite, carborundum, steel including stainless steel, copper, aluminum and ceramics. In some embodiments the material is in a form in which its outer surface area is large including small spheres, cylinders, rings, filament, meshes and ribbons and the like. In one embodiment, a reaction zone A″ contains from 0.1 to 5 percent by weight of at least one catalyst capable of effecting the oxidation of propylene to acrolein and/or the oxidation of acrolein to acrylic acid.

It may be desirable to pack the catalyst or catalysts into the tubes of the reactor or reactors in a manner so as to provide a particular peak-to-salt temperature sensitivity. The packing methods for creating reaction zones having differing activities described previously hereinabove may be used, for example, to achieve a particular peak-to-salt temperature sensitivity, as is well understood by persons of ordinary skill in the art. For example, it may be beneficial to pack the catalyst or catalysts into the tubes so as to achieve a peak-to-salt temperature sensitivity of not more than 9° C., such as, for example, not more than 6° C., or not more than 3° C. As used herein, the “peak-to-salt temperature sensitivity” means the increase in catalyst peak temperature, measured in ° C., which is brought about by increasing the heat transfer medium temperature by 1° C.

It is noted that the salt-to-peak temperature sensitivity is a characteristic of the particular packed tube and catalyst configuration. The peak-to-salt temperature sensitivity of a particular packed tube configuration may be empirically determined by testing the in situ packed tube in the reaction system, or testing another packed tube which is equivalent to the packed tube utilized in the oxidation reactor (i.e., having the same tube size (internal and external diameters) and cross-section, the same material of construction, the same length, etc.), and which is packed in the same manner as the in situ packed tube in the oxidation reactor (using or not using zones such as A, A′, A″, B, B′ and B″, etc., having the zone(s) of the same length as contemplated for actual reactor operation, having the zone(s) contain the same catalyst(s) as contemplated for actual use) and fitted out with heat transfer zone(s) as the same manner as the in situ packed tube of interest. In either case, the peak-to-salt temperature sensitivity is empirically determined by raising the temperature of the heat transfer medium and measuring the corresponding increase of the catalyst peak temperature and then calculating the increase of the catalyst peak temperature for each 1° C. in temperature increase of the heat transfer medium. Moreover, as would be understood by persons of ordinary skill in the art, as an operational approximation, the relationship between the catalyst concentration in the tube and the catalyst temperature increase resulting from increases in the salt temperature, is assumed to be linear. Thus, the salt-to-peak temperature sensitivity of a particular packed tube may be adjusted by varying the catalyst concentration, as described in detail hereinabove in connection with the R1 and R2 catalyst formulations and, typically, no more than one or two, or at most three, tests are required to adjust the peak-to-salt temperature sensitivity to be within the desired range.

In an alternative embodiment of the process of the present invention (not shown), the multiple catalyst system of the present invention is used with at least two tandem reactors wherein the first stage reactor includes a reaction zone A which contains one or more R1 catalysts at least capable of catalyzing oxidation of propylene to acrolein and the second stage reactor includes a reaction zone B which contains one or more R2 catalysts at least capable of catalyzing oxidation of acrolein to acrylic acid. Further details regarding the design of tandem reactor systems is found in EP 1070 700 A2 which is hereby incorporated herein in its entirety. In a typical embodiment, a tandem reactor system is used wherein the first stage reactor includes reaction zones A and A′ which each contain one or more R1 catalysts at least capable of catalyzing oxidation of propylene to acrolein and the second stage tandem reactor includes reaction zones B and B′ which each contain one or more R2 catalysts at least capable of catalyzing oxidation of acrolein to acrylic acid. In one embodiment, reaction zones A and A′ have a different catalytic activity for converting propylene into acrolein and/or reaction zones B and B′ have a different activity for converting acrolein to acrylic acid. As recited above, several means are available for achieving differing catalytic activities.

Typically, reactive hydrocarbon is fed to a single reactor system reactor at a RHSV of 75 to 300 hr⁻¹. One embodiment employs a reactive hydrocarbon feed rate of 135 to 250 hr⁻¹, another embodiment utilizes 140 to 230 hr⁻¹.

When a tandem reactor system is used, the reactive hydrocarbon is typically fed to a first oxidation reactor of a tandem reactor system of at a RHSV of 75 to 300 hr⁻¹ or 135 to 250 hr⁻¹, or 135 to 225 hr⁻¹ and then on to a second oxidation reactor of the tandem system at a RHSV of 75 to 300 hr⁻¹, or 135 to 250 hr⁻¹, or 135 to 225 hr⁻¹.

In the tandem reactor configuration, the initial RHSV to the second oxidation reactor is calculated based on the total volume of the initial reactant feed, including the propylene concentration, and the volume (unpacked) of the second oxidation reactor tubes.

The total space velocity, TSV of a fluid (e.g., gas phase process stream) through the R1 and R2 catalysts ranges from about 75 hr⁻¹ to 5000 hr⁻¹, with 500 hr⁻¹ to about 3000 hr⁻¹ being typical. In one embodiment, utilizing a 10% concentration of propylene feed gas to a 25,000 tube reaction system, the TSV of the process gas is about 1000 hr⁻¹ when the propylene feed rate to the reaction system is about 20,000 lb/hr of propylene, the TSV of the process gas is about 1800 hr⁻¹ when the propylene feed rate to the reaction system is about 35,000 lb/hr of propylene, and the TSV of the process gas is about 2500 hr⁻¹ when the propylene feed rate to the reaction system is about 50,000 lb/hr of propylene. On a propylene basis, this is equivalent to a C3-SV of 102 hr⁻¹, 178 hr⁻¹, and 255 hr⁻¹, respectively.

In one embodiment, utilizing an 8% concentration of propylene feed gas to a 25,000-tube reaction system, the TSV of the process gas is about 1300 hr⁻¹ when the propylene feed rate to the reaction system is about 20,000 lb/hr of propylene, the TSV of the process gas is about 2500 hr⁻¹ when the propylene feed rate to the reaction system is about 40,000 lb/hr of propylene, and the TSV of the process gas is about 3800 hr⁻¹ when the propylene feed rate to the reaction system is about 60,000 lb/hr of propylene. On a propylene basis, this is equivalent to a C3-SV of 102 hr⁻¹, 204 hr⁻¹, and 306 hr⁻¹, respectively.

In another embodiment, utilizing a 7% concentration of propylene feed gas to a 25,000-tube reaction system, the TSV of the process gas is about 1500 hr⁻¹ when the propylene feed rate to the reaction system is about 20,000 lb/hr of propylene, the TSV of the process gas is about 1800 hr⁻¹ when the propylene feed rate to the reaction system is about 25,000 lb/hr of propylene, and the TSV of the process gas is about 2500 hr⁻¹ when the propylene feed rate to the reaction system is about 35,000 lb/hr of propylene. On a propylene basis, this is equivalent to a C3-SV of 102 hr⁻¹, 127 hr⁻¹, and 178 hr⁻¹, respectively.

The heat transfer medium of a reactor having an R1 or R2 catalyst disposed therein may circulate in any manner deemed suitable for the particular reactor system utilized. Tube and baffle arrangements in tube fixed bed shell reactors which provide for cocurrent, countercurrent, transverse and bypass flows of the heat transfer medium may be employed. The baffles may be arranged so as to have equal spacing between baffles or variable spacing between baffles.

The operational maximum (“peak”) R1 and R2 catalyst temperatures are controlled to be in a range which is typically 20° C. to 70° C. above the heat transfer medium temperature. The heat transfer medium may be any heat transfer medium suitable for use under the temperature conditions of the process of the present invention, which is typically between 300° C. and 500° C. Generally the heat transfer medium is a salt melt, which is a typical heat transfer medium for use acrylic acid production processes. One embodiment of the process of the present invention utilizes a salt melt of 40 wt % to 80 wt % potassium nitrate and 60 wt % to 20 wt % sodium nitrate. Another embodiment uses 50 wt % to 70 wt % percent by weight potassium nitrate and 50 wt % to 30 wt % sodium nitrite. Sodium nitrate can be substituted for sodium nitrite, or potassium nitrate or employed as an additional component of the salt melt. Sodium nitrate is typically present at up to 20 wt % and one embodiment employs a concentration of sodium nitrate which is about 10 wt % of the total salt composition. Additional, optional, heat transfer mediums include, but are not limited to, heat transfer oils, both oleaginous and synthetic, and phenyl ethers and polyphenyls, and low melting metals e.g., sodium, tin, mercury, low melting metal alloys.

In one embodiment of the process of the present invention employed for acrylic acid production, increasing the heat transfer medium temperature by 1° C. has been found to increase the peak catalyst temperature by 2-3° C. In practice, as catalyst loses activity with age, or run-time, reaction temperature is increased to control reaction rates and capacities.

The heat transfer medium may circulate within the reactor in at least two distinct heat transfer medium circuits. In one embodiment, there is more than one heat transfer medium circuit and at least one circuit is associated with each heat transfer zone corresponding to a reaction zone. EP 1070 700 A2 discloses further details regarding the design of tandem reactor systems and their heat transfer mechanisms.

In one embodiment 98% conversion of propylene to acrolein is achieved utilizing an R1 catalyst in a first reaction zone and an initial salt temperature of about 325° C. The 98% conversion rate is maintained by raising the cold salt temperature slowly to 345° C., at which point, the reactor system may be taken offline to allow for regeneration of the catalyst. After regeneration of the catalyst, which may take from 12-24 hours as discussed in further detail hereinafter, 84%-98% conversion is again obtained with a salt temperature of 328° C. or higher. Because catalyst regeneration does not provide complete recovery of yield—that is, the resultant regenerated catalyst efficiency is generally less than 100% of the efficiency of fresh (new) catalyst—the foregoing cycle of operation and catalyst regeneration may be repeated until the obtained yield recovery is so small as to make it no longer economically advantageous to continue operating the catalyst system. This point will vary depending on market conditions, however, typically, it will no longer be economically advantageous to continue operating the catalyst system when catalyst regeneration does not increase yield by more than about 0.1%. At this point the used R1 catalyst may be replaced with fresh, new R1 catalyst.

One embodiment of the process of the present invention utilizes a multiple catalyst system comprising at least one R1 catalyst and at least one R2 catalyst to produce acrylic acid from propylene. The AA yield achieved by the process of the present invention is typically from 80% to 95%, for example, without limitation, from 85%-90%. Furthermore, the process of the present invention, when utilized for acrylic acid production, provides service life of the catalyst system (and reaction process, or reaction system) of 2 years or more, with periodic regeneration of the R1 catalyst, as will be described in further detail hereinafter. In this regard, it is noted that the individual useful life of the R1 catalyst may be extended by regeneration to up to 6 years or more. It is also noted that, while the R2 catalyst is not adversely affected by exposure to oxygen containing gas during regeneration of the R1 catalyst, the R2 catalyst is not itself regenerated to a significant degree and, therefore, it must be periodically replaced with a new R2 catalyst charge (i.e., up to about 2 years, which provides the limiting factor on the life of the overall multiple catalyst system). Nonetheless, it has been found that the life of the multiple catalyst system of the present invention, when utilized in connection with one or more of the reaction processes described hereinabove, is longer than the service life of other known catalyst systems. The utilization of the multiple catalyst system of the present invention, including R1 and R2 catalysts described hereinabove, in one or more processes as disclosed herein results in yields that are about 1-1.5% higher yield in acrylic acid production, as compared to R1 or R2 catalysts independently and not used in series or in combination, or different R1 and R2 type catalysts not in conformity with the catalyst definitions provided hereinabove.

Typically, the reactive hydrocarbon is present in the initial reactor feed composition at a range from 1 to 20 vol % percent based upon the total volume of the feed stream. For example, the reactive hydrocarbon is an unsaturated hydrocarbon, such as, but not limited to, propylene, isobutylene and mixtures thereof. In one embodiment, the reactive hydrocarbon is a hydrocarbon derivative including, but not limited to, acrolein, (meth)acrolein and mixtures thereof. It is noted that the reactive hydrocarbon may be from any source, including but not limited to, a fresh feed stream, recycled from other portions of the reaction process, or product or side streams from other processes.

In one embodiment, the reactive hydrocarbon is propylene, and is present in a range of from 4 to 11 vol %. In another embodiment, the reactive hydrocarbon is present in a range of 7 vol % to 11 vol % based on the total volume of the feed stream. In a further embodiment, the reactive hydrocarbon is present in a range 7.5 vol % to 9 vol % based on the total volume of the feed stream.

In embodiments where the reactive hydrocarbon is acrolein, the acrolein may be from any source and may be any grade suitable for an acrylic acid producing vapor phase oxidation reaction. Typically, the acrolein is generated by and supplied from the first stage reaction zone of the process for the production of acrylic acid from propylene described above. The acrolein may be produced in situ in the first stage of a single reactor system and be introduced into the second stage of the single reactor system for conversion to acrylic acid.

Alternatively, the acrolein may be generated in a first oxidation reactor of a tandem reactor system and then passed into a second oxidation reactor as part of the second reactant composition for conversion into acrylic acid. Acrolein is generally present in a reactant composition at a range from 1 vol % to 22 vol % of the reactant composition. In one embodiment, acrolein is present at a range of from 5 vol % to 18 vol %. In another embodiment acrolein is present at a range of 6 vol % to 12 vol % of the reactant composition.

It is noted that where the process of the present invention is performed using a tandem reactor system, it may be a “staged air reactor” system, which would include additional air, or other oxygen-containing gas, being introduced to one or more stages, vessels, or reaction zones downstream (after) the first stage, vessel or reaction zone. It is noted, the oxygen concentration in the feed to the first stage reaction zone of a staged air reactor is typically lower than the oxygen concentration in the feed to an SRS or a non-staged tandem reactor system, and the resulting propylene concentration in the feed to the first stage reaction zone of a staged air reactor system is, therefore, higher (e.g., optionally from 0.5-10% higher, or more) than the propylene concentration in the feed to the second stage reaction zone.

U.S. Pat. No. 6,620,968 discloses the details of a process for producing unsaturated aldehydes and acids from reactive hydrocarbons by vapor phase catalytic oxidation at high reactive hydrocarbons space velocities and its entire contents are hereby incorporated herein by reference. Processes in accordance with the present invention, utilizing the multiple catalyst system with either an SRS reactor or a tandem reactor system will now be described in further detail.

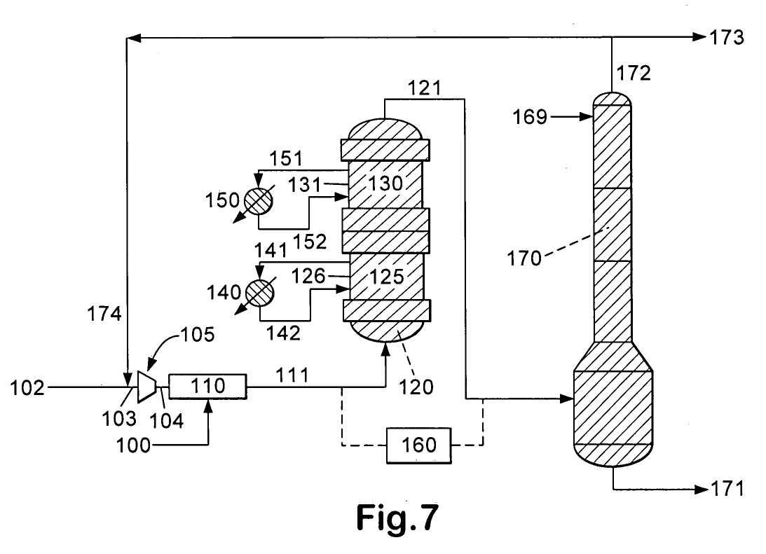

FIG. 7 illustrates an embodiment of the process of the present invention which utilizes the above-described the multiple catalyst system and an SRS reactor to produce acrylic acid from propylene by two-step vapor phase catalytic oxidation. This process combines an oxygen containing gas stream (102) and, optionally, a recycle stream (174), to form a compressor feed stream (103). A compressor exit stream (104) leaves the compressor (105) and is combined with a propylene feed stream (100) by mixer (110). The propylene feed (100) stream comprises any source and any grade of propylene feed suitable for a vapor phase oxidation reaction, as described previously hereinabove. In a typical embodiment, the propylene source is chemical grade propylene.

In one embodiment, the oxidation of propylene to acrylic acid utilizes a propylene concentration in the reactor feed gas composition of between 4 and 7 vol % based on the total reactor feed composition with a suitable space velocity.

Typically, the propylene feed stream of an SRS reactor is generally less than 15 wt % propylene. One embodiment utilizes 7-8 wt % propylene. In a staged air system embodiment the propylene feed stream is typically in a range of about 10-15 wt % propylene. Where propylene is utilized, it is generally present in the reactant composition at greater than 7 vol % of the reactant composition. It is noted that the reactive hydrocarbon space velocities, RHSV's, of the process of the present invention may be suitably increased by decreasing the concentration of the propylene in the feed and concurrently increasing the feed rates.

The oxygen containing gas stream (102) may comprise pure oxygen, air, or any reaction-compatible oxygen containing gas. Examples of suitable constituents of the oxygen containing gas stream (102) are, without limitation, air, oxygen-enriched air, pure oxygen, and mixtures of pure oxygen and at least one inert gas or mixtures thereof. A typical source of oxygen is air. Generally, the amount of oxygen containing gas which is fed to the process should be sufficient to deliver an amount of molecular oxygen which will provide an oxygen/propylene ratio in the reactant composition of from 1.6:1.0 to 2.2:1.0, such as, for example, from 1.6:1.0 to 2.0:1.0.

The reactor feed stream composition(s) may also contain water vapor, which may be present in the reactant composition in an amount of from about 2 to 30 vol %, or 5 to 11 vol %, of the reactor feed composition. Alternatively, in the absence of recycle, the water vapor may be present at a range from 15 to 25 vol. % of the reactant composition. The water vapor may be provided by recycle from other process steps or may be otherwise generated and provided to the reactant composition, or it may be provided by both recycle and generation.

Inert gas may be included in the reactor feed composition and may comprise any gaseous material, or mixtures of gaseous materials, which is inert to the oxidation reactions of the present invention. Typical examples include, but are not limited to, nitrogen, carbon dioxide, helium, argon, propane and carbon monoxide, or mixtures thereof. One embodiment of the process of the present invention employs nitrogen as the inert gas, or a mixture of nitrogen with at least one other inert gas. Suitable sources of inert gases include, for example, nitrogen or air, recycled absorber off-gas, recycled stack gas, and mixtures thereof. When obtained from recycle streams, the inert gas generally constitutes a major amount of the remainder of the reactant composition which is not propylene, oxygen, or water vapor. Typically, the inert gas is 50 to 99.9 vol %, for example, without limitation, 60 to 99.9 vol % of the remainder of the reactant composition.

As recited above, the reaction composition may optionally include at least one inert gas which is suitable for use as fuel for thermal or catalytic oxidation/incineration of waste absorber off-gas. Such inert gas fuel may be provided as part of the impurities in the propylene feed, as part of the absorber off-gas, or as neat chemical. Generally, such inert gas fuel is present in a minor amount in the reactant composition. In one embodiment, the inert gas fuel is less than 15 vol % of the reactant composition. In another the inert gas feed is less than 10 vol % of the reactant composition. When carbon oxides and inert gas fuel are present in the reactant composition, the combined amount thereof is typically less than 15 vol % of the reactant composition, and may be less than 10 vol % of the reactant composition.

In one embodiment, the water vapor, an inert gas and, optionally, at least a portion of the inert gas fuel of the reaction composition, are provided by recycle of at least a portion the absorber off-gas (“AOG”) to the reactor.

Generally, in acrylic acid manufacturing processes the amount of AOG recycled is a sufficient source of water vapor and inert gas. However, the absorber off-gas is not required (or may not be available in sufficient quantities to) provide all of the requirements of water vapor and/or inert gas of the system. Thus, additional amounts of water and/or inert gas may be added from other sources.

When AOG recycle is not used, steam and nitrogen are used as the primary diluents. Steam is not consumed, but may alter the selectivity, conversion and/or catalytic activity in the oxidation reactions and is part of the mixed product gases emerging from the reactor.

The compressor (105) is downstream of (i.e., after) a “tee” which joins the air feed stream (102) and, optionally, the absorber off gas (AOG) recycle stream (174) and is located upstream (before) of the mixer (110). The reactor feed stream (111) is fed to the SRS reactor (120).

As described hereinabove, the amount of propylene present in the reactor feed composition suitable for use in the process of the present invention may be between 4.0 and 11.0 vol % based on the total volume of reactant feed composition, and is typically greater than 7 vol % of the reactant composition. In one embodiment, for example, propylene is present in a range of reactant composition of 7.0 to 11.0 vol %. In still another embodiment, propylene is present in a range of from 7.0 to 9.0 vol % in the reactant feed composition and in a further embodiment it is between 7.5 and 9.0 vol % of the total reactant feed composition. It is noted that the propylene concentration in the gas feed composition may even be between 4.0 and 7.0 vol % of the total reactant feed composition.

The SRS reactor (120) comprises a first reaction zone (125) and a second reaction zone (130), each having tubes (not shown per se) passing therethrough and being separated by a perforated tube sheet (similar to perforated tube sheet (3) shown in FIG. 1). It should be understood that the SRS reactor (120) may comprise additional reaction zones (not shown), as described above in connection with FIGS. 3-5. In the first reaction zone (125), the reactor feed gas is brought into contact with at least one R1 catalyst disposed in tubes (not shown) of the first reaction zone (125) in a manner similar to that described hereinabove in connection with the reaction zones A, A′, A″ shown in FIGS. 3-5. The gaseous product stream of the first reactor zone (125) passes by the perforated tube sheet (not shown in FIG. 7) and enters the second reaction zone (130) as feed gas. As the feed gas passes through the second reaction zone (130), it is brought into contact with at least one R2 catalyst disposed in the tubes (not shown) of the second reaction zone (130) in a manner similar to that described hereinabove in connection with the reaction zones B, B′, B″ shown in FIGS. 3-5 and is, thereby, converted to a reactor exit gas stream (121).

The temperature of the first reaction zone (125) is thermally controlled by a first heat exchanger (140), which establishes the first heat transfer zone (126) across the first reaction zone (125). Heat transfer medium is fed to the first heat exchanger (140) by a first heat exchanger feed stream (141) and returned by a first heat exchanger return stream (142).

A second heat exchanger (150) establishes a second heat transfer zone (131) across the second reaction zone (130). Heat transfer medium is fed to the second heat exchanger (150) by a second heat exchanger feed stream (151) and returned by a second heat exchanger return stream (152).

The reactor exit gas stream (121) may be fed to an absorber (170), which produces aqueous acrylic acid stream (171) and absorber off-gas stream (AOG) (172). In the absorber (170), some reaction by-products, including acetic acid, formaldehyde, maleic acid and other organics, are absorbed along with the acrylic acid into the aqueous acrylic acid stream (171). Unreacted propylene, most of the unreacted acrolein, inert fuel gas such as propane, CO₂, CO, and N₂ are not absorbed and leave the absorber (170) as AOG (172). The AOG (172) may further comprise minor amounts of acrylic acid and other byproducts of the reaction. The AOG (172) may be conveyed to a thermal/catalytic oxidizer or incinerator. Alternatively, the AOG (172) can be recycled to the reactor as described above.

With reference still to FIG. 7, operation of the SRS reactor (120) and the process conditions of the overall process may be controlled, in part, by the utilization of yield and selectivity data. In a one embodiment, for example, a process analyzer (160) can be utilized in the process to collect and analyze samples of the reactor gas stream (121) and provide the resulting sample data to calculate comparative yield and/or selectivity data. Suitable analyzer types include but are not limited to mass spectrometers (MS), gas chromatographs (GC), Fourier transform infra red (FTIR) analyzers, and the like. More particularly, the resulting process analyzer sample data may be used to calculate the concentrations of CO₂, or acrolein, acrylic acid or any other constituent of reactor feed stream (111) and reactor exit gas stream (121). Differences in yields or selectivity over time may be employed to make control decisions in the operation of the SRS reactor (120).

The AOG stream (172), is optionally split to provide an AOG recycle stream (174) and an absorber off-gas waste stream (173). An absorber feed stream (169) is feed to the top of the absorber (170) and typically comprise water, or another suitable solvent, as is well known to persons of ordinary skill in the art. The AOG recycle stream (174) is optionally fed through a heater (not shown) that is positioned close (i.e., optionally within fifty meters) to the outlet of the absorber (170). A stream (not shown) from a thermal or catalytic oxidizer may optionally be combined with the air feed (102). Alternatively, nitrogen or steam (not shown) may be combined with the air feed (102) in addition to, or instead of, the AOG recycle stream (174). In one embodiment, the AOG waste stream (173) is optionally fed to a thermal oxidizer or a catalytic oxidizer.

The process of the present invention optionally includes a unit operation for stripping light ends from the aqueous acrylic acid solution (171) of the absorber (170). In one embodiment, the concentration of acrylic acid in the stripped aqueous acrylic acid solution is in a range of 55 wt % to 85 wt %. In another embodiment, the concentration of acrylic acid in the stripped aqueous acrylic acid solution 60 wt % to 80 wt %.

The absorption may take place by methods known in the art, such as feeding the mixed product gas comprising acrylic acid up to an absorption tower and feeding a stream comprising water as the absorbent. The mixed product gas comprising acrylic acid generally is fed to the absorption tower typically at a temperature of from 200° C. to 400° C. One embodiment utilizes a temperature range of 250° C.-350° C. The temperature in the absorption tower generally ranges from 30° C. to 100° C., with one embodiment utilizing a temperature range of 40° C. to 80° C. The ratio of water fed to the absorption tower to the reactor propylene feed rate ranges from 0.35:1 to 1, with one embodiment utilizing a ratio of about 0.35.

In one embodiment, water vapor in reactor feed stream (111) is at least in part provided by recycle of the AOG (174) back to the reactor.

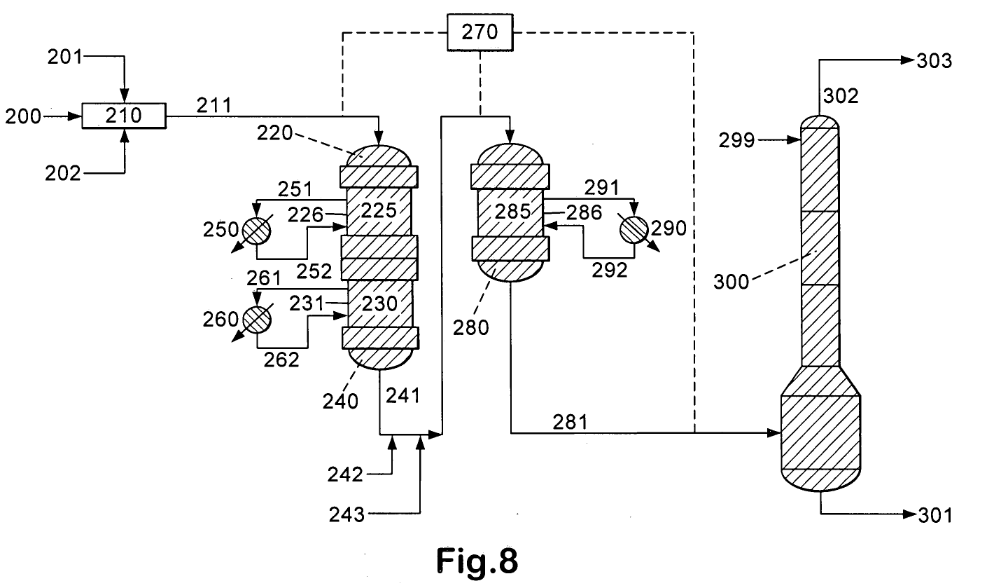

With reference now to FIG. 8, in an alternative embodiment of the process of the present invention, as mentioned hereinabove, the multiple catalyst system may be employed in a tandem reactor system which includes two reactors, i.e., one for each stage, for performing the two-step oxidation of propylene to acrylic acid. In such a system, the first reactor is utilized to produce acrolein which is then passed through an interstage cooler and on to a second reactor wherein the acrolein is oxidized to acrylic acid. It should be understood that it is not necessary that the second reactor be operated in tandem with the first reactor or any another reactor. Rather, the second reactor may stand alone and produce an unsaturated aldehyde or acid.

In one embodiment, the inventive process comprises a first stage reactor having an interstage portion. It is optional whether embodiments employing an interstage portion will sustain reaction in the interstage portions. Some embodiments do not sustain reaction in the interstage portion.

A typical tandem reactor process design has one or more feed streams (200), (201), (202), as shown in FIG. 8, from which the reaction composition is formed that is fed to the process. Examples of suitable feed streams for use with this embodiment of the present invention, include, without limitation, a propylene feed stream (201), an oxygen containing gas feed stream (200), and an inert stream (202).

The initial reaction composition may further include at least one inert gas which may be suitable for use as fuel for thermal oxidation/incineration of waste AOG. Such inert gas fuel may be provided as part of the impurities in the propylene feed, as part of the absorber offgas, or as the neat chemical. Typical inert gases include, but are not limited to, nitrogen, carbon dioxide, helium, argon, propane, ethane, methane, butane, pentane or mixtures thereof. One embodiment employs propane as an inert gas. Another embodiment utilizes an inert gas including nitrogen, or a mixture of nitrogen with at least one other inert gas. The inert gas generally constitutes a major amount of the remainder of the reactant composition which is not propylene, oxygen, or water vapor. In one embodiment, the inert gas concentration is 50 to 99.9 vol %. In other embodiments, the inert gas concentration is 60 to 99.9 vol % of the remainder of the reactant composition. The water vapor in the reaction composition is generally present at a range from 10 to 40 vol %, and in one embodiment is 15 to 25 vol % of the reactant composition.

Generally, such inert gas fuel is present in a minor amount in the remainder of the reactant composition which does not include propylene, oxygen and water vapor. Generally, inert gas fuel comprises 0.001 to 49.9 vol %, and in one embodiment ranges from 0.1 to 20 vol % of the total reactant feed composition.

The oxygen containing gas stream (200) may comprise pure oxygen, air, or any reaction-compatible oxygen containing gas. Examples of suitable constituents of the oxygen containing gas stream (200) are, without limitation, air, oxygen-enriched air, pure oxygen, and mixtures of pure oxygen and at least one inert gas or mixtures thereof. A typical source of oxygen is air. Generally, the amount of oxygen containing gas which is fed to the process should be sufficient to deliver an amount of molecular oxygen which will provide an oxygen/propylene ratio in the reactant composition of from 1.6:1.0 to 2.2:1.0, such as, for example, from 1.6:1.0 to 2.0:1.0.

The inert feed stream (202) comprises inert gases, which may include one or more of AOG, N₂, CO₂, CO, or other non-reactive inert gases, and mixtures thereof. The inert gases may be provided to the process individually, or mixed, and may be direct feeds, or part of a recycle process. The various feed streams (201), (200) and (202) are combined by a mixer (210) to form reactor feed stream (211). The reactor feed stream (211) is fed to a first reaction section (220). The first reaction section (220) comprises a first reaction zone (225) and catalyzes the first stage of catalytic reaction in the production of acrylic acid to produce acrolein. This first stage of reaction is catalyzed in the first reaction zone (225) by at least one R1 catalyst disposed therein. A first heat transfer zone (226) is associated with the first reaction zone (225) for controlling the temperature of the first reaction zone (225). The first heat exchanger (250) utilizes heat transfer medium fed by a first heat exchanger feed stream (251) and returned by a first heat exchanger return stream (252). The reaction effluent from the first reaction section (220) passes to the interstage section (240). Interstage section (240), as discussed above, may be optionally connected or disconnected from reaction section (220). The interstage section (240) comprises interstage zone (230). The interstage zone (230) may comprise inert material, or catalyst, or may simply provide heat exchange surface area with no inert or catalyst present. A second heat transfer zone (231) is associated with the interstage zone (230) for controlling the temperature of the interstage zone (230). A second heat exchanger (260) is fed by a second heat exchanger feed stream (261) and returns the heat transfer medium via a second heat exchanger return stream (262). Both of the first and second heat exchangers (250) and (260) may employ a molten salt heat transfer media.

More particularly, the interstage zone (230) is typically, but is not required to be, a non-reactive zone and provides holdup, residence time, interstage cooling and surface area sufficient to minimize migration of molybdenum from the R1 catalyst downstream of interstage zone (230) and into the second reaction zone (285). The effluent of the interstage section (240) is first stage exit gas stream (241). The first stage exit gas stream (241) may be fed to the second reaction zone (285) through an optional, additional heat exchanger (not shown in FIG. 8). Further, the first stage exit gas stream (241) may be combined with an oxygen containing gas (242) and/or an inert gas stream (243).

The first stage exit gas stream (241) is fed to second stage reaction section (280). Reaction section (280) comprises the second reaction zone (285). In one embodiment, the second reaction section (280) contains at least one R2 catalyst. The second reaction zone (285) has an associated third heat transfer zone (286) comprising a third heat exchanger (290), for controlling the temperature of the second reaction zone (285). The third heat exchanger (290) is fed by a third heat exchanger feed stream (291) and the heat transfer medium is returned by a third heat exchanger return stream (292).

The second reaction section (280) produces a second stage exit gas stream (281) (also know as “reactor gas stream”), which is fed to an absorber (300). The second stage exit gas stream (281) may optionally be fed to another heat exchanger (not shown in FIG. 8). Further, the second stage exit gas stream (281) can be utilized as a source of thermal cross-exchange, or as a pre-heat stream, for other streams of the production process. One embodiment, for example, may use the second stage exit stream (281) to provide heat transfer to streams (202), (243), and optionally stream (303).

When an aqueous absorbent is used, absorber (300) produces an aqueous acrylic acid stream (301). Where a non-aqueous absorbant is used, stream (301) may be an acrylic acid non-aqueous solution. An absorbent feed stream (299) optionally comprises either water, or other absorbents, or a combination of absorbents, as are well known to persons of ordinary skill in the art. The absorber (300) produces an AOG overhead stream (302). The AOG stream (302) may be further processed as an AOG waste stream (303) comprising inert gases, oxygen, water vapor, CO, CO₂, unreacted propylene, unreacted acrolein and/or acrylic acid. The AOG waste stream (303) may be fed to a thermal oxidizer or catalytic oxidizer or catalytic combustion unit. The AOG stream (302) may form an AOG recycle stream (not shown), which is fed to the reactor feed stream (211) and/or first stage exit gas stream (241).

The reactors of a tandem reactor oxidation process may stand alone connected by appropriate piping systems, or can be combined as different portions (sections, compartments) within the same pressure vessel. The reactors can be arranged with a first stage coupled to an interstage section (as shown in FIG. 8), or an interstage section coupled to a second stage reactor (not shown). Where the interstage section (240) is connected to the second reaction section (280), the exit gas stream (241) of the first reaction section (220) may be combined with an oxygen containing gas, or an inert gas stream, upstream of the interstage section (240). Generally, each of these stages has an associated heat transfer system. There is no limitation to the number of such stages that a tandem reactor system suitable for use in connection with the present invention may posses, nor is the arrangement necessarily limited or critical, but rather these factors depend on economic and reaction factors as will be readily understood by persons of ordinary skill in the art.

It will also be recognized by those skilled in the art that the reactor may also be maintained at two or more temperature ranges, as noted above, and that two or more heat transfer circuits may be utilized for each reactor.

Control of the processes of the present invention are achieved in a variety of ways. Without limitation, stream compositions, temperatures and pressure are exemplary factors in control. Process analyzers, such as those previously described, are typically employed to assist in the monitoring of existing conditions and possible desirable modifications to the aforesaid factors.

As described in connection with the embodiment of FIG. 7 hereinabove, the SRS reactor is, in part, controlled by the utilization of yield and selectivity data obtained by process analyzers, such as, for example, a gas chromatograph or mass spectrometer.

As shown in FIG. 8, the control system for the tandem reactor system may utilize a process analyzer (270). The process analyzer (270) is, for example, connected to any type of control or distributed control system. The process analyzer (270) receives data by sampling process streams such as the reactor feed stream (211) and/or the first stage exit gas stream (241) and/or the second stage exit gas stream (281). The control system may be utilized to sample additional streams of the process as well, and can also receive samples from the reactors' interstage sections and/or any piping system or point in the process, which one having ordinary skill in the art desires to take a sample. In one embodiment, for example, the process analyzer is a gas chromatograph. The process analyzer (270) may also be a mass spectrometer or any such device suitable for analyzing gas samples. Control data may include, without limitation, one or more sample values of concentrations of CO₂, acrolein, propylene, acrylic acid, CO, and acetic acid.

In acrylic acid processing the intermediate stream comprising acrolein is not typically isolated before going to the second reaction section (280), but it may be isolated or treated. For example, in some embodiments of the process of the present invention, it may be desirable to cool the intermediate stream comprising acrolein and/or add oxygen containing gas and/or recycle streams between the first and second reaction sections (220), (280).

As is generally understood in the art, reaction systems employing R1 and R2 type catalysts experience higher temperatures and higher conversion at the expense of selectivity over time. Inversely, the selectivity of an R1 and R2 type catalyst system rises over time, as propylene conversion decreases and reactor temperature is decreased. Increases in the product yield can be achieved by raising the reactor operating temperature over 300° C., or by conducting a regeneration of the R1 catalyst. The time required for sufficient regeneration of the R1 catalyst is typically 12 hrs-24 hrs.

The salt temperature of the heat transfer zones of a process in accordance with the present invention may be adjusted to control product yields. More specifically, the operating temperatures of the at least one R1 catalyst, and of the multiple catalyst system generally, ranges from about 250° C. to about 500° C. One embodiment of the process of the present invention, for example, maintains the temperature of the at least one R1 catalyst in a range of from about 300° C. to about 400° C.

It is noted that, in practice, to avoid, or at least minimize, over-utilization, or “burn-out”, of the multiple catalyst system, the process of the present invention may be operated under operating conditions which result in less that 100% conversion of the reactive hydrocarbon (for example, without limitation, less than 100% propylene conversion, or less than 100% isobutylene conversion). More particularly, the process of the present invention may be operated at a reactive hydrocarbon space velocity of from 135 hr⁻¹ to 250 hr⁻¹, at multiple catalyst system temperatures of from 300° C. to 400° C., and reactive hydrocarbon feed concentrations of from 7.0 vol % to 11.0 vol %, to produce propylene conversion in a range from 90% to less than 100% and an acrolein conversion (i.e., in the second stage reaction zone(s)) of in a range from 90% to less than 100%. Propylene conversion may be, for example, but not limited to, in a range of from 94% to 99%, or even from 95% to 98%. Acrolein conversion may be, for example, but not limited to, in a range of from 95% to 99.9%, or even from 97% to 99.7%.

Processes in accordance with the present invention, which comprise the multiple catalyst system of the present invention, may operate more than 500 hours without requiring air purging of the catalyst system or catalyst regeneration. For example, in one embodiment, the at least one R1 catalyst does not require air purging for more than about 800 hours.

The values of conversion and selectivity normally vary over time in reaction systems where the inventive catalyst system is employed. More particularly, it is not unusual to see a decrease in the yield of acrylic acid of about 0.8% for each 1,000 hours of run-time. More particularly, it has been found that the acrylic acid yield loss is typically about 0.4% per 1,000 hours of run-time. To compensate for such acrylic acid yield losses, over the 1,000 hours of run-time, the salt temperature of the heat transfer zones may be raised from about 1° C. to about 25° C.

Raising the salt temperature of the reactor is one method that may improve yield and reduce loss of conversion. Thus, as the yield drops over time, the raise of the salt temperature by a difference of +25° C. over a run cycle can increase the run-time (i.e., the useful life) of the multiple catalyst system in a reactor by anywhere from 1 to 1,000 hours. In one embodiment, the salt temperature and, therefore the temperature of the reactor, may be raised from 1° C. to 25° C., at a rate of from 0.5° C. to 2° C., per 100 hours of run-time. In another embodiment, the salt temperature and, therefore, the temperature of the reactor, may be increased by 1° C. to 2° C. per 100 hours of run-time. In yet another embodiment, a 1.5° C., per 100 hour increase of the salt temperature is employed.

There are additional methods of recapturing lost yields as the multiple catalyst system, and particularly the R1 catalyst, loses activity through use, besides the adjustment of the salt and reactor temperatures. More particularly, the conversion and selectivity of the R1 catalyst of the multiple catalyst system of the present invention is improved by purging the reactor with an oxygen containing gas such that the catalyst is exposed to the oxygen containing gas for some period of time, as will be described in further detail hereinafter. The passage of an oxygen containing gas through an R1 or R2 catalyst provides a “regeneration” of catalytic activity and, therefore, this process is generally known as catalyst “regeneration”. It is noted that the regeneration of an R1 and/or an R2 catalyst may be achieved by the passage of any oxygen containing gas stream over the catalyst, including but not limited to air, steam, a steam and air mixture, or a gas stream comprising oxygen. Air is an economical and effective selection for the oxygen containing gas.

It is well known that oxidation catalysts may be degraded when exposed to excessive temperatures, which is likely to result in lower yield and reduced catalyst service life. The extent to which a given catalyst is thermally degraded will vary, and may depend on various factors such as the actual exposure temperature, the catalyst composition, and the duration of high-temperature exposure. R1 catalysts used in the conversion of propylene to acrolein, as in the present invention, are typically degraded at least to some degree, but generally not irreversibly, at temperatures in excess of 350° C. R2 catalysts used in the conversion of acrolein to acrylic acid, as in the present invention, are typically degraded at least to some degree, but generally not irreversibly, at temperatures in excess of 300° C. To avoid such degradation, a maximum service temperature limit is generally employed for processes which include R1 and R2 catalysts, such as the multiple catalyst system of the present invention. For commercially available R1 and R2 catalysts, the recommended maximum service temperature may be obtained from the catalyst producer. Alternatively, users of such catalysts, as well as producers of such catalysts, may determine the maximum service temperatures of the R1 and R2 catalysts through simple operating-temperature-versus-yield experimentation, which will be readily understood by persons having ordinary skill in the art. It has been found that the maximum service temperature limit for R1 catalysts in accordance with the present invention is about 415° C. and the maximum service temperature limit for R2 catalysts is about 350° C.

In view of the foregoing circumstances, when the R1 catalyst of the multiple catalyst system of the present invention is regenerated, it will be apparent to one of ordinary skill in the art that it is desirable to limit the temperature of the oxygen containing gas to which the R1 catalyst is exposed to a temperature that does not exceed the recommended maximum service temperature of the catalyst(s) being contacted.

In one embodiment of the process of the present invention wherein catalyst regeneration is performed, the oxygen containing gas used is an air feed stream at a temperature between 105° C. and 415° C. In another embodiment, the air feed stream temperature is between 275° C. and 400° C., such as between 340° C. to 360° C. In a further embodiment, an air feed stream temperature of between 175° C. and 220° C., such as 200° C., is utilized. A typical heat transfer salt temperature during reactor regeneration (regardless of whether an SRS reactor or a tandem reactor system is employed) is approximately 300° C. to 450° C. One embodiment employs reactor temperatures between 325° C. and 400° C.

Regeneration of an R2 catalyst may utilize an oxygen containing gas stream, such as but not limited to air, at a temperature of between 275° C. and 350° C. For example, a temperature of 248° C. is used in one embodiment, and another embodiment uses a range of 300° C.-325° C.

Since the maximum service temperature of the R2 catalyst is typically less than the maximum service temperature of the R1 catalyst, the regeneration fluid (i.e., the oxygen-containing gas) used to regenerate the R1 catalyst should be prevented from contacting the R2 catalyst while it is at a temperature greater than the maximum service temperature of the R2 catalyst. More particularly, for example, in one embodiment of the process of the present invention wherein the multiple catalyst system is employed in an SRS reactor of the type generally depicted in FIG. 7, the temperature of the oxygen-containing gas contacting the R1 catalyst is maintained at or below 350° C. to avoid degradation of the R2 catalyst positioned in series and downstream of the R1 catalyst. In an alternative embodiment of the process of the present invention wherein the inventive catalyst system is employed in a tandem reactor system, with or without staged air, of the type generally depicted in FIG. 8, the temperature of the oxygen-containing gas used to regenerate the R1 catalyst may be allowed to reach up to 415C, provided that steps are taken to prevent exposure of the R2 catalyst to excessive temperatures. Such steps may include, but are not limited to: cooling the oxygen containing gas after contacting the R1 catalyst to a temperature of not more than 350° C. in one or more heat exchangers before it contacts the R2 catalyst; blending the oxygen containing gas, after it has contacted the R1 catalyst, with a second low-temperature fluid, such as for example process air, to produce a blended stream with a temperature of not more than 350° C. before it contacts the R2 catalyst; and redirecting the flow of the oxygen containing gas after contacting the R1 catalyst, such that contact with the R2 catalyst is substantially avoided.

The oxygen containing gas may be fed to the reaction system such that it contacts at least the R1 catalyst for a period of time, known as the “regeneration period”, of greater than 1 hour, such as, between 1 hour and 48 hours. For example, without limitation, a regeneration period of 12 hours may be used. The time necessary to accomplish regeneration of the multiple catalyst system of the present invention may be measured by the yield recovery and selectivity increases determined by the percentage of acrylic acid in the reactor gas stream by analytical methods discussed hereinabove, including but not limited to, the use of a gas chromatograph. For example, it has been found that a regeneration period of 12-24 hours provides a yield recovery of between 50% and 95%. It has been observed in a process for producing acrylic acid from propylene, utilizing the multiple catalyst system of the present invention, that 12 hours of heated air exposure provides recovery of up to 90% of the lost acrylic acid yield.

The run-time between performing regeneration of the multiple catalyst system may be between about 800 hours and 1,000 hours. However, it should be understood that catalyst regeneration may be conducted as frequently as desired.

It is noted that, in practice, the catalyst activity (i.e., reactive hydrocarbon conversion) increases, but selectivity to the desired unsaturated aldehyde or acid product drops initially following a catalyst regeneration period. Thus, operation immediately after regeneration is much like the break-in period (also known as the “conditioning period”) for a new catalyst charge, wherein a period of on-stream time under reaction conditions is required for catalyst selectivity to return to normal. In addition, while the employment of a longer regeneration period may ultimately result in better product yield recovery, there are drawbacks including, but not limited to, longer regeneration period which also means a longer off-stream (unproductive) period, as well as a longer conditioning period following the regeneration before the maximum yield is regained. It is noted that the conditioning period may vary from 24 hours to as much as a week, depending on how active the catalyst is made during the catalyst regeneration process. Furthermore, the use of higher temperatures for the oxygen containing gas used for the regeneration have a directionally similar impact, however, the off-stream (unproductive) period is minimized when the regeneration is performed at relatively higher temperatures (for example, at 360° C. rather than at 220° C.).

The regeneration procedure may be monitored and controlled, for example, by the measurement of CO₂ in reactor exit gas stream (121) of FIG. 7 or stream (241) of FIG. 8. Regeneration control based on other streams may also be utilized. During the regeneration cycle, when the CO₂ concentration falls to acceptable levels, the air feed may be discontinued and process startup operations may commence. It is common to discontinue air feed when the CO₂ concentration in reactor gas stream (121) or stream (241) decreases to a concentration of about equal to or less than 1.2 times the ambient air concentration of CO₂., or alternatively, to a concentration that is within the statistical tolerance of the measuring equipment indicating a negligible or non-existent CO₂ concentration. The measure of CO₂ during regeneration allows for higher temperatures of the oxygen containing gas and of the heating medium (e.g., molten salt) to be employed during the regeneration process thereby shortening the regeneration cycle.

In one embodiment, the oxygen-containing gas is blown into the reaction system with a blower, or, alternatively, oxygen-containing gas flow may be achieved using a compressor. The blower, or compressor, is employed to overcome the pressure drop of the reactor system. In a one embodiment, using an SRS reactor, an oxygen-containing gas feed pressure at the reactor inlet of between 10 psig and 20 psig is employed. However, regeneration may be achieved at pressures from slightly above atmospheric pressure to the designed pressure of the vessel, for example, 0-150 psig, or greater. In one embodiment, of the reactor system, the compressor is employed to overcome the pressure drop of a reactor having 25,000 tubes and a designed pressure of 50 bar. In another embodiment, a compressor is utilized to produce 25 psig of pressure at the reactor inlet, yielding 135 pounds per hour of an air mixture.

It has been found that the commercially useful operating life of an R1 catalyst in accordance with the present invention is greater than the commercially useful operating life of an R2 catalyst in accordance with the present invention, under normal operating conditions of the process of the present invention, regardless of whether an SRS reactor or a tandem reactor system is used. An R1 catalyst typically has a life of 1-10 years under normal operating conditions, which include those discussed hereinabove with periodic regeneration at about every 1,000 to 2,000 run-time hours. In some embodiments, 3-6 years is the operating life of the R1 catalyst. The life of an R1 catalyst is typically greater than two years. The typical life of an R2 catalyst is from one to five years. An R2 operating life of 1.5 to 2 years is associated with some embodiments and greater than 0.5 years is typically expected.

Even with the employment of higher operating temperatures and catalyst regeneration procedures, the overall life expectancies of the R1 catalyst and R2 catalysts are finite. In typical operation, the R1 and R2 catalyst of the multiple catalyst system of the present invention are each replaced at a time which is determined on a case by case basis generally based upon evaluations of diminishing yield or selectivity. As will be readily recognized by persons having ordinary skill in the art, such determinations may be made based upon a number of additional factors, including but not limited to, product market conditions, cost of replacement catalysts, time constraints, and the like. It is noted that the R1 and R2 catalysts of the multiple catalyst system need not be, and typically are not, replaced at the same time and that the replacement R1 and R2 catalysts are typically of the same general type as the spent catalyst being replaced (i.e., R1 and R2 catalysts, respectively).

It has been found that, using the multiple catalyst system of the present invention in a reaction system having an SRS reactor, acrylic acid can be produced under stable conditions and in high yield at reactive hydrocarbon space velocities of greater than 75 hr⁻¹. Moreover, regeneration of the multiple catalyst system, as discussed hereinabove, using air at 330° C. to 350° C. over regeneration periods of 12-24 hours, results in the maintenance of high acrylic acid yields over relatively long periods of time, for example, in excess of 4,000 hours.

It will be understood that the embodiments of the present invention described herein are merely exemplary and that a person skilled in the art may make variations and modifications without departing from the spirit and scope of the invention. All such variations and modifications are intended to be included within the scope of the present invention.

### EXAMPLES

To demonstrate the advantages and improvements achieved by the multiple catalyst system and the process of the present invention, a pilot reaction system employing an SRS type reactor was used to convert propylene to acrylic acid by vapor phase catalytic oxidation, under similar process conditions, but using four different catalyst systems, as follows.

Reactor Configuration and Reaction Conditions

The same pilot reaction system apparatus was used for the oxidation process of all four examples provided hereinbelow. More particularly, the reaction system apparatus included an SRS type of reactor having stainless steel catalyst contact tubes of approximately 1-inch diameter. Similar to the configurations shown and described in connection with FIGS. 2, 4 and 5 hereinabove, each tube of the SRS type reactor had a first stage reaction section with at least one reaction zone, a second stage reaction section with at least one reaction zone, and an intermediate quenching zone positioned in between the first and second reaction sections. Each of the first and second stage reaction sections had a heat transfer zone associated therewith, each of which utilized molten salt as the heat transfer medium.

Each tube also included a first layer of diluent material positioned immediately upstream of the first stage reaction section and a second layer of diluent material positioned immediately downstream of the second stage reaction zone, as well as diluent material in the intermediate quenching zone. More particularly, the diluent material was stainless steel Raschig rings having a nominal length of 6 mm and a nominal outer diameter of 6.5 mm.

Depending on the manner in which the catalysts were packed into the tubes, which will be described in further detail in connection with each of the following examples hereinafter, the first stage reaction section always included at least a first reaction zone A and sometimes also a second reaction zone A′. Similarly, the second stage reaction section always included at least a first reaction zone B and sometimes also a second reaction zone B′. It is noted that the catalysts were packed into the tubes such that, as described hereinabove in connection with the process of the present invention, peak-to-salt sensitivity was less than 9° C. for both the first stage and the second stage reaction sections.

For each of the following four examples, the feed composition contained about 7% by volume of chemical grade propylene, as well as an amount of air and absorber off-gas sufficient to maintain an oxygen/propylene ratio of about 1.8 to 1.9, and a water vapor concentration of about 10% by volume. This was provided to the first stage reaction section of the SRS type reactor at a reactive hydrocarbon space velocity (RHSV) of about 102 hr⁻¹.

The temperature of the molten salt associated with the heat transfer zone of the first stage reaction zone was maintained between about 300° C. and 360° C. The temperature of the molten salt associated with the heat transfer zone of the second stage reaction section was maintained between about 275° C. and 295° C.

Catalyst Preparations

Catalyst R1*: A suitable R1 catalyst, i.e., in accordance with the present invention, was prepared by a method equivalent to the following process:

In 1 liter of ion-exchange water, 550 g of cobalt nitrate, 412 g of nickel nitrate and 286 g of ferric nitrate were dissolved, and 92 g of bismuth nitrate was dissolved in aqueous nitric acid consisting of 50 g of 61 wt. % nitric acid and 200 ml of ion-exchange water. Separately, 1000 g of ammonium paramolybdate and 25 g of ammonium paratungstate were added to 3 liters of heated ion-exchange water, and dissolved under stirring. Into thus formed aqueous solution, the two aqueous solutions which were separately prepared were added dropwise and mixed, and into the mixture then an aqueous solution of 3.8 g of potassium nitrate in 50 ml of ion-exchange water and 141 g of silica sol of 20 wt. % in concentration were added by the order stated. Thereafter 178 g of basic bismuth nitrate (product of Kanto Chemical Co.) was added to the mixture to provide a slurry containing the following elements: Mo, W, Bi, Fe, Co, Ni, K and Si. The molar ratio of the total nitrate anions to the molybdenum [NO₃]/[Mo] in this slurry was 1.8.

This slurry was heated under stirring, evaporated to day solid and dried. The resulting solid was pulverized and molded into rings of each 6 mm in outer diameter, 2 mm in inner diameter and 6.6 mm in length, which were calcined at temperatures between 450° C. and 530° C., for 6-8 hours in an air stream.

The metal element composition of this catalyst (R1*) was substantially as follows (in terms of atomic ratio excepting oxygen, as in all of the following Examples):
Mo₁₂W_{0.2}Bi_{1.7}Fe_{1.5}Co₄Ni₃K_{0.08}Si₁
Catalyst R2*: A suitable R2 catalyst, i.e., in accordance with the present invention, was prepared by a method equivalent to the following process:

In a first formulating tank, equipped with a stirring motor, 600 parts of 95° C. deionized water and 16.26 parts of ammonium tungstate were put and stirred. 18.22 parts of ammonium metavanadate and 110 parts of ammonium molybdate were then added to dissolve. 7.75 parts of antimony acetate was added further. After 20 min, 2.07 parts of niobium oxide was added. In 96 parts of deionized water held in a second formulating tank, 15.05 parts of copper nitrate was dissolved and the resultant solution was added into the first formulating tank to get a slurry solution.

The slurry solution was spray-dried under adjusting the feed rate so that the temperature at the outlet of a spray drier might be kept at about 100° C. to get a dried granule. The granule thus obtained was calcined at temperatures between 370° C. and 400° C. for about 5 hrs in a furnace, of which the temperature had been risen at a rate of about 60° C. per hour from the room temperature.

The preliminary calcination granule thus obtained was pulverized in a ball mill to get a preliminary calcination powder. 12 parts of the preliminary calcination powder thus obtained was homogeneously mixed with 0.77 parts of crystalline cellulose and re-calcined at temperatures between 370° C. and 400° C. for about 5 hrs in a furnace.

The atomic ratio of a catalytically active component excluding oxygen in the coated catalyst thus obtained is as follows:
Mo₁₂V₃W_{1.2}Cu_{1.2}Sb_{0.5}Nb_{0.3}
Catalyst “CA”: An R2 type catalyst of composition which did not conform to the R2 type catalyst of the present invention, was prepared by a method equivalent to the following process:

In 2,500 ml of water being heated and stirred were dissolved 350 g of ammonium paramolybdate, 116 g of ammonium metavanadate and 44.6 g of ammonium paratungstate. Thereto was added 1.5 g of vanadium trioxide. Separately, 87.8 g of copper nitrate was dissolved in 750 ml of water being heated and stirred, after which 1.2 g of cuprous oxide, 12 g of antimony trioxide arid 2.2 g of stannous oxide were added thereto. The resulting two fluids were mixed and placed in a porcelain-made evaporator set on a hot water bath. Thereto was added 1,000 ml of a spherical .alpha.-alumina carrier having diameters of 3-5 mm. The mixture was evaporated to dryness with stirring to adhere the above compounds onto the; carrier, followed by firing at 400° C. for 6 hours, to obtain a catalyst having the following composition (excluding oxygen):
Mo₁₂V_{6.1}W₁Cu_{2.3}Sb_{0.5}Sn_{0.1}
Catalyst CD: An R1 type catalyst of composition which did not conform to the R1 type catalyst of the present invention, was prepared by a method equivalent to the following process:

An aqueous solution (A) was obtained by dissolving 423.8 g of ammonium molybdate and 2.02 g of potassium nitrate in 3,000 ml of distilled water which was heated and stirred.

Separately from this, an aqueous solution (B) was prepared by dissolving 302.7 g of cobalt nitrate, 162.9 g of nickel nitrate and 145.4 g of ferric nitrate in 1,000 ml of distilled water, and an aqueous solution (C) by dissolving 164.9 g of bismuth nitrate in 200 ml of distilled water which had been acidified by adding 25 ml of concentrated nitric acid. The aqueous solutions (B) and (C) were mixed, and the mixture solution was added dropwise to the aqueous solution (A) which was vigorously stirred.

The thus formed suspension was dried using a spray dryer and subjected to 3 hours of preliminary calcination at 440° C., thereby obtaining 570 g of preliminarily calcined powder. Thereafter, 200 g of the preliminarily calcined powder was mixed with 10 g of crystalline cellulose as a molding additive, and thus a mixture of the preliminarily calcined powder and the crystalline cellulose was obtained.

A 300 g portion of alumina carrier having an average particle size of 3.5 mm was put into a tumbling granulator and then the just described mixture and 90 g of 33% by weight glycerol aqueous solution as a binder were simultaneously added thereto to effect support of the mixture on the carrier, thereby obtaining particles having a supported ratio of 40% by weight (to be referred to as active component-supported particles hereinafter).

The active component-supported particles were dried at room temperature for 15 hours and then calcined at a temperature between 520° C. and 560° C. for 5 hours in the flow of air to obtain a catalyst having an average particle size of 4.0 mm, and a the composition of the catalytically active components (excluding oxygen) was as follows:
Mo₁₂Bi_{1.7}Ni_{2.8}Fe_{1.8}Co_{5.2}K_{0.1}

### Example 1

The aforesaid pilot reaction system was loaded with an embodiment of the multiple catalyst system of the present invention and used to convert propylene to acrylic acid under the process conditions provided above with the following additional modifications.

The tubes of the SRS type reactor were packed with an R1 catalyst and an R2 catalyst such that the first stage reaction section had a first and a second reaction zone A and A′, and the second stage reaction section had a first and a second reaction zone, B and B′, as follows. In the first stage reaction section, the R1* catalyst described above and which conformed to the present invention was loaded into first and second reaction zones A and A′ (see, e.g., FIG. 4). In the second stage reaction section, the R2* catalyst described above and which conformed to the present invention was loaded into reaction zones B and B′ (see, e.g., to FIG. 4). The R1* and R2* catalysts in zones A and B, respectively, were both diluted by mixing them with inert DENSTONE 57° catalyst bed supports (available from Norton Chemical Products Corp., of Akron, Ohio) such that the first reaction zone A contained a mixture of 66 wt % of the R1* catalyst and 34% DENSTONE 57®, and the first reaction zone B contained a mixture of 75 wt % of the R2* catalyst and 25% DENSTONE 57®. Each of the second reaction zones A′ and B′ contained 100 wt % of the R1* catalyst and the R2* catalyst, respectively. The total lengths of the first stage reaction section and the second stage reaction section were both between 3000 mm and 4000 mm. The first reaction zone A accounted for about the first one-third of the length of the first stage reaction section and the second reaction zone A′ accounted for the remaining two-thirds of the length of the first stage reaction section. Similarly, the first reaction zone B accounted for about the first one-third of the length of the second stage reaction section and the second reaction zone B′ accounted for the remaining two-thirds of the length of the second stage reaction section. These catalyst dilutions constrained peak-to-salt sensitivity to less than 9° C. for both the first stage and the second stage reaction sections.

Acrylic acid formation reactions were carried out over a trial period of more than 2,000 hours and salt temperature was regularly adjusted to obtain acrylic acid yield with propylene conversion of greater than 95%.

It is noted that when the first stage reaction section peak temperature rose to about 390° C. (which occurred approximately every 700-1000 hours during the trial runs), the process feeds were stopped and a catalyst regeneration procedure was performed. The catalyst regeneration procedure was conducted for a period of from 24 to 72 hours, and involved providing a purge gas stream, comprising air and between 5 and 10% water vapor, to the catalyst tubes at a purge gas space velocity of 1000 hr⁻¹, while maintaining the temperature of the molten salt associated with the heat transfer zone of the first stage reaction section between 330° C. and 350° C. Upon completion of the regeneration process, feeds were re-introduced into the SRS reactor tubes, the temperature of the molten salt was reduced by between 1° C. and 10° C., and catalyst conversion and selectivity were allowed to stabilize for about 100 hours before yield measurements were resumed.

The propylene concentration (% C3), the ratio of oxygen to propylene (O2/C3), and the concentration of water vapor (% steam) in the reactor feed stream were maintained essentially constant throughout the trial period. AA was produced in significant yields.

### Comparative Example A

The aforesaid pilot reaction system was loaded with a multiple catalyst system that was not in accordance with the present invention, wherein a suitable R1 catalyst was used, along with a non-conforming R2 type catalyst, to convert propylene to acrylic acid under process conditions equivalent to those used for Example 1, with the following additional modifications.

The tubes of the SRS type reactor were packed with an R1 and an R2 catalyst such that the first stage reaction section had a first and a second reaction zone A and A′, and the second stage reaction section had a first and a second reaction zone, B and B′, as follows. In the first stage reaction section, the suitable R1* described above was loaded into first and second reaction zones A and A′ (see, e.g., FIG. 4). In the second stage reaction section, the CA catalyst (a non-conforming R2 type catalyst) described above was loaded into reaction zones B and B′ (see, e.g., to FIG. 4). The R1* and CA catalysts in zones A and B, respectively, were both diluted by mixing them with inert DENSTONE 57® catalyst bed supports (available from Norton Chemical Products Corp., of Akron, Ohio) such that the first reaction zone A contained a mixture of 66 wt % of the R1* catalyst and 34% DENSTONE 57®. The first reaction zone B contained a mixture of 87 wt % of the CA catalyst and 13% DENSTONE 57®. The second reaction zone A′ of the first reaction section contained 100 wt % of the R1* catalyst. The second reaction zone B′ of the second reaction section contained 100 wt % of the CA catalyst. The total lengths of the first stage reaction section and the second stage reaction section were both between 3000 mm and 4000 mm. The first reaction zone A accounted for about the first one-third of the length of the first stage reaction section and the second reaction zone A′ accounted for the remaining two-thirds of the length of the first stage reaction section. Similarly, the first reaction zone B accounted for about the first one-third of the length of the second stage reaction section and the second reaction zone B′ accounted for the remaining two-thirds of the length of the second stage reaction section. These catalyst dilutions constrained peak-to-salt sensitivity to less than 9° C. for both the first stage and the second stage reaction sections.

Acrylic acid formation reactions were carried out over a trial period of more than 2,000 hours and salt temperature was regularly adjusted to obtain acrylic acid yield with propylene conversion of greater than 95%.

The propylene concentration (% C3), the ratio of oxygen to propylene (O2/C3), and the concentration of water vapor (% steam) in the reactor feed stream were maintained essentially constant throughout the trial period. The AA yield of this Comparative Example A was about 2% less than the AA yield achieved in Example 1.

### Comparative Example C

The aforesaid pilot reaction system was loaded with a multiple catalyst system that was not in accordance with the present invention, wherein a non-conforming R1 type catalyst was used, along with a suitable R2 catalyst, to convert propylene to acrylic acid under process conditions equivalent to those used for Example 1, with the following additional modifications.

The tubes of the SRS type reactor were packed with an R1 and an R2 catalyst such that the first stage reaction section had only a first reaction zone A, and the second stage reaction section had a first and a second reaction zone, B and B′, as follows. In the first stage reaction section, the CD catalyst (a non-conforming R1 type catalyst) described above was loaded into the first reaction zone A (see, e.g., FIG. 2). In the second stage reaction section, the suitable R2* catalyst described above was loaded into the first and second reaction zones B and B′ (see, e.g., to FIG. 4). The CD catalyst in reaction zone A was loaded without any diluent material. Thus, the first reaction zone A of the first reaction section contained 100 wt % of the CD catalyst. The R2* catalyst in reaction zone B was diluted by mixing it with inert DENSTONE 57® catalyst bed supports (available from Norton Chemical Products Corp., of Akron, Ohio) such that the first reaction zone B of the second stage reaction section contained a mixture of 75 wt % of the R2* catalyst and 25% DENSTONE 57®. The second reaction zone B′ of the second reaction section contained 100 wt % of the R2* catalyst. The total lengths of the first stage reaction section and the second stage reaction section were both between 3000 mm and 4000 mm. The first reaction zone B accounted for about the first one-third of the length of the second stage reaction section and the second reaction zone B′ accounted for the remaining two-thirds of the length of the second stage reaction section. The foregoing catalyst packing schedules constrained peak-to-salt sensitivity to less than 9° C. for both the first stage and the second stage reaction sections.

Acrylic acid formation reactions were carried out over a trial period of more than 2,000 hours and salt temperature was regularly adjusted to obtain acrylic acid yield with propylene conversion of greater than 95%.

The propylene concentration (% C3), the ratio of oxygen to propylene (O2/C3), and the concentration of water vapor (% steam) in the reactor feed stream were maintained essentially constant throughout the trial period. The AA yield of this Comparative Example C was initially similar to the AA yield achieved in Example 1, but after about 250 hours of operation, the AA yield dropped to more than about 1.5% less than the AA yield achieved in Example 1.

We claim:

1. A multiple catalyst system, comprising:
at least one first catalyst and at least one second catalyst which are capable of catalyzing the oxidation of a reactive hydrocarbon to its corresponding unsaturated carboxylic acid,
said at least one first catalyst being capable of being regenerated by exposure to an oxygen containing gas and being capable of catalyzing the oxidation of the reactive hydrocarbon to at least a second reactive hydrocarbon and having a composition expressed by the general formula:
MoₐBi_{b}Fe_{c}A_{d}EₑOₓ,
wherein O is oxygen;
A is at least one element selected from among Ni and Co; E is at least an element selected among alkali metal elements or alkaline earth metal elements, Tl, P, Te, Sb, Sn, Ce, Pb, Nb, Mn, As, Zn, Si, B, Al, Ti, Zn and W; and
wherein a, b, c, d, e and x are the relative atomic ratios of the respective elements Mo, Bi, Fe, A, E and O, where a is 12, b is 0.1-10, c is 0.1-20, d is 1-20, e is 0-30, and x is a positive numerical value determined by the oxidation state of the other elements; and
said at least one second catalyst being capable of maintaining its activity levels upon exposure to an oxygen-containing gas and being capable of catalyzing the oxidation of the second reactive hydrocarbon to the corresponding unsaturated carboxylic acid and having a composition expressed by the general formula:
MoₐV_{b}Cu_{c}(W)_{d}(Sb)ₑ(A)_{f}(G)_{g}(Y)ₕOₓ;
wherein A is at least an element selected from among alkali metal elements, and thallium;
G is at least one element selected from among alkaline earth metals and zinc;
Y is at least one element selected among Nb, Mn, Fe, Co, Ge, Sn, As, Ce, Ti, and Sm;
O is oxygen; and
wherein a, b, c, d, e, f, g, h, and x are the relative atomic ratios of the respective elements Mo, V, Cu, W, Sb, A, G, Y and O, where a is 12, b is 0.5-12, c is less than or equal to 6, d is 0.2-10, e is positive and less than or equal to 10; f is 0-0.5; g is 0-1; h is positive and less than 6; and x is a positive numerical value determined by the oxidation state of the other elements.

2. The multiple catalyst system according to claim 1, wherein at least one of said at least one first catalyst comprises Mo, Bi, Fe and Ni.

3. The multiple catalyst system according to claim 1, wherein at least one of said at least one second catalyst comprises Mo, V, Cu, W and Sb.

4. The multiple catalyst system according to claim 1, wherein at least one of said at least one first catalyst is diluted to a range of 50 wt % to 80 wt %.

5. The multiple catalyst system according to claim 1, wherein the reactive hydrocarbon is propylene, the second reactive hydrocarbon is acrolein, and the unsaturated carboxylic acid is acrylic acid.

6. A method of regenerating a catalyst system, comprising the steps of:
providing a multiple catalyst system disposed in a reactor apparatus employed in the oxidation of a reactive hydrocarbon to its corresponding unsaturated carboxylic acid, said catalyst system comprising at least one first catalyst and at least one second catalyst, which are capable of catalyzing the oxidation of the reactive hydrocarbon to the corresponding unsaturated carboxylic acid,
said at least one first catalyst being capable of being regenerated by exposure to an oxygen containing gas and being capable of oxidizing the reactive hydrocarbon to at least a second reactive hydrocarbon and having a composition expressed by the general formula:
MoₐBi_{b}Fe_{c}A_{d}EₑOₓ,
wherein O is oxygen;
A is at least one element selected from among Ni and Co; E is at least an element selected among alkali metal elements or alkaline earth metal elements, Tl, P, Te, Sb, Sn, Ce, Pb, Nb, Mn, As, Zn, Si, B, Al, Ti, Zn and W; and
wherein a, b, c, d, e and x are the relative atomic ratios of the respective elements Mo, Bi, Fe, A, E and O, where a is 12, b is 0.1-10, c is 0.1-20, d is 1-20, e is 0-30, and x is a positive numerical value determined by the oxidation state of the other elements; and
said at least one second catalyst being capable of maintaining its activity levels upon exposure to an oxygen-containing gas and being capable of oxidizing the second reactive hydrocarbon to the corresponding unsaturated carboxylic acid and having a composition expressed by the general formula:
MoₐV_{b}Cu_{c}(W)_{d}(Sb)ₑ(A)_{f}(G)_{g}(Y)ₕOₓ;
wherein A is at least an element selected from among alkali metal elements, and thallium;
G is at least one element selected from among alkaline earth metals and zinc;
Y is at least one element selected among Nb, Mn, Fe, Co, Ge, Sn, As, Ce, Ti, and Sm;
O is oxygen; and
wherein a, b, c, d, e, f, g, h, and x are the relative atomic ratios of the respective elements Mo, V, Cu, W, Sb, A, G, Y and O, where a is 12, b is 0.5-12, c is less than or equal to 6, d is 0.2-10, e is positive and less than or equal to 10; f is 0-0.5; g is 0-1; h is positive and less than 6; and x is a positive numerical value determined by the oxidation state of the other elements;
oxidizing the reactive hydrocarbon by exposing the reactive hydrocarbon to the multiple catalyst system in the presence of oxygen until the yield of unsaturated carboxylic acid decreases by more than from 0.5% to 10%;
discontinuing the oxidation of the reactive hydrocarbon and the oxidation of the second reactive hydrocarbon; and
exposing said at least one first catalyst to an oxygen containing gas at a temperature ranging from at least 105° C. to less than or equal to 415° C.

7. The method according to claim 6, wherein the exposing step is performed for a period of from 1 to 48 hours.

8. The method according to claim 6, further comprising the step of preventing the oxygen-containing gas from contacting said at least one second catalyst while the oxygen-containing gas is at a temperature greater than 350° C.

9. A catalytic vapor phase oxidation process, comprising:
providing a first oxidation reactor comprising a plurality of tubes disposed in a reactor shell having an interior, the interior of the reactor shell being divided into at least a first heat transfer zone through each of which a heat transfer medium passes; each of said tubes containing at least one first catalyst, said at least one first catalyst being packed in a manner so as to provide a peak-to-salt temperature sensitivity of not more than 9° C., said at least one first catalyst being capable of being regenerated by exposure to an oxygen containing gas and having a composition expressed by the general formula:
MoₐBi_{b}Fe_{c}A_{d}EₑOₓ,
wherein O is oxygen;
A is at least one element selected from among Ni and Co; E is at least an element selected among alkali metal elements or alkaline earth metal elements, Tl, P, Te, Sb, Sn, Ce, Pb, Nb, Mn, As, Zn, Si, B, Al, Ti, Zn and W; and
wherein a, b, c, d, e and x are the relative atomic ratios of the respective elements Mo, Bi, Fe, A, E and O, where a is 12, b is 0.1-10, c is 0.1-20, d is 1-20, e is 0-30, and x is a positive numerical value determined by the oxidation state of the other elements; and
feeding said first reactant composition comprising
(i) at least one first reactive hydrocarbon, and
(ii) oxygen
into said first oxidation reactor, at a first reactive hydrocarbon space velocity of from 135 hr⁻¹ to 300 hr⁻¹, to contact said first reactant composition with at least one first catalyst to form a first product gas comprising at least one second reactive hydrocarbon and oxygen;
wherein, when each said tubes of said first oxidation reactor comprises a plurality of sequentially disposed reaction zones, each reaction zone after the first reaction zone of each of said tubes has a temperature that is less than 5° C. greater than its immediately preceding reaction zone;
providing a second oxidation reactor comprising a plurality of tubes disposed in a reactor shell, the inside of the reactor shell being divided into at least a first heat transfer zone through each of which a heat transfer medium passes; each of said tubes containing at least one second oxidation catalyst, said at least one second oxidation catalyst being capable of maintaining its activity levels upon exposure to an oxygen-containing gas and being capable of effecting the oxidation of said second reactive hydrocarbon and oxygen to a final product gas comprising (meth)acrylic acid and having a composition expressed by the general formula:
MoₐV_{b}Cu_{c}(W)_{d}(Sb)ₑ(A)_{f}(G)_{g}(Y)ₕOₓ;
wherein A is at least an element selected from among alkali metal elements, and thallium;
G is at least one element selected from among alkaline earth metals and zinc;
Y is at least one element selected among Nb, Mn, Fe, Co, Ge, Sn, As, Ce, Ti, and Sm;
O is oxygen; and
wherein a, b, c, d, e, f, g, h, and x are the relative atomic ratios of the respective elements Mo, V, Cu, W, Sb, A, G, Y and O, where a is 12, b is 0.5-12, c is less than or equal to 6, d is 0.2-10, e is positive and less than or equal to 10; f is 0-0.5; g is 0-1; h is positive and less than 6; and x is a positive numerical value determined by the oxidation state of the other elements, said tubes containing at least one second oxidation catalyst being packed with said at least one second catalyst in such a manner so as to provide a peak-to-salt temperature sensitivity of not more than 9° C.;
feeding said first product gas comprising
(i) at least one second reactive hydrocarbon, and
(ii) oxygen
into said second oxidation reactor, at a second reactive hydrocarbon space velocity of from 135 hr⁻¹ to 300 hr⁻¹; to contact said first product gas with said at least one second oxidation catalyst to form a final product gas comprising (meth)acrylic acid;
wherein, when each said tube of said second oxidation reactor comprises a plurality of sequentially disposed reaction zones, each reaction zone after the first reaction zone of each of said tubes has a temperature that is less than 5° C. greater than its immediately preceding reaction zone.
